(19) United States
(12) Patent Application Publication
(10) Pub. No.: US 2025/0352336 A1
(43) Pub. Date: Nov. 20, 2025

(54) PROSTHETIC HEART VALVE

(71) Applicant: EDWARDS LIFESCIENCES CORPORATION, Irvine, CA (US)

(72) Inventors: Roy Shitrit, Zichron Yaakov (IL); Nikolai Gurovich, Hadera (IL)

(73) Assignee: EDWARDS LIFESCIENCES CORPORATION, Irvine, CA (US)

(21) Appl. No.: 19/286,695

(22) Filed: Jul. 31, 2025

Related U.S. Application Data
(63) Continuation of application No. PCT/US2024/017412, filed on Feb. 27, 2024.
(60) Provisional application No. 63/448,767, filed on Feb. 28, 2023.

(51) Int. Cl. A61F 2/24

(52) U.S. Cl. CPC A61F 2/2418; A61F 2230/0004

(57) ABSTRACT

A prosthetic valve includes a frame and a leaflet assembly having a plurality of leaflets coupled to the frame. The leaflet assembly is movable between an open state and a closed state to allow unidirectional blood flow through the prosthetic valve. Each leaflet includes an outflow edge, a cusp edge, and a pair of commissure tabs extending from opposite sides of the leaflet and between the outflow edge and the cusp edge. Each commissure tab is paired with an adjacent commissure tab of an adjacent leaflet to form a commissure of the leaflet assembly. The outflow edge of each leaflet includes a coaptation edge region and cut-out regions between opposite ends of the coaptation edge region and the commissure tabs. When the leaflet assembly moves between the open and closed states, the coaptation edge regions pivot relative to the commissure tabs at respective pivot axes intersecting the cut-out regions.

### CROSS-REFERENCE TO RELATED APPLICATIONS

This application is a continuation of International Patent Application No. PCT/US2024/017412, filed Feb. 27, 2024, which claims the benefit of U.S. Provisional Application No. 63/448,767, filed Feb. 28, 2023, all of which applications are incorporated by reference herein in their entireties.

### FIELD

The present disclosure relates to implantable, radially expandable prosthetic devices, such as prosthetic heart valves, and to methods, assemblies, and apparatuses for delivering, expanding, implanting, and deploying such prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve (or simply “prosthetic valve”) can be mounted in a crimped state on the distal end of a delivery apparatus and advanced through the patient's vasculature (for example, through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

Despite the recent advancements in percutaneous valve technology, there remains a need for improved transcatheter prosthetic valves.

### SUMMARY

The present disclosure relates to methods and devices for treating valvular diseases. Specifically, the present disclosure is directed to implantable, radially expandable prosthetic devices, such as prosthetic heart valves, and to methods, assemblies, and apparatuses for delivering, expanding, implanting, and deploying such prosthetic devices.

A prosthetic valve can include a radially expandable and compressible frame comprising an inflow end and an outflow end, and a leaflet assembly comprising a plurality of leaflets coupled to the frame. In addition to these features, a prosthetic valve can further comprise one or more of the components disclosed herein.

In certain examples, the leaflet assembly is movable between an open state which permits blood flow from the inflow end to the outflow end and a closed state which blocks blood fluid flow from the outflow end to the inflow end.

In certain examples, each leaflet can include an outflow edge, a cusp edge opposite to the outflow edge, and a pair of commissure tabs extending from opposite sides of the leaflet and between the outflow edge and the cusp edge.

In certain examples, each commissure tab can be paired with an adjacent commissure tab of an adjacent leaflet to form a commissure of the leaflet assembly that is connected to the frame.

In certain examples, the outflow edge of each leaflet can include a coaptation edge region and cut-out regions between opposite ends of the coaptation edge region and the commissure tabs.

In certain examples, when the leaflet assembly moves between the open and closed states, the coaptation edge regions can pivot relative to the commissure tabs at respective pivot axes intersecting the cut-out regions.

In certain examples, the outflow edge of each leaflet is formed with a notch adjacent each commissure tab.

In certain examples, the outflow edge of each leaflet includes a coaptation edge region and chamfered or rounded edges connecting the coaptation edge region to the corresponding commissure tabs.

In certain examples, each commissure tab includes an outflow edge portion which extends closer to the outflow end than the coaptation edge region of the corresponding leaflet.

According to certain aspects of the disclosure, a method of assembling a prosthetic valve can include receiving a radially expandable and compressible frame comprising an inflow end and an outflow end, receiving a plurality of leaflets, forming a leaflet assembly, and attaching commissures to the frame. Each leaflet includes an outflow edge, a cusp edge opposite to the outflow edge, and a pair of commissure tabs extending from opposite sides of the leaflet and between the outflow edge and the cusp edge. The outflow edge of each leaflet includes a coaptation edge region and cut-out regions between opposite ends of the coaptation edge region and the commissure tabs. Forming the leaflet assembly includes pairing each commissure tab with an adjacent commissure tab of an adjacent leaflet to create a corresponding commissure. The attaching maintains the cut-out regions within an interior space of the frame so that the coaptation edge regions are spaced radially apart from an inner surface of the frame.

According to certain aspects of the disclosure, a method of assembling a prosthetic valve can include receiving a radially expandable and compressible frame including an inflow end and an outflow end; receiving a leaflet assembly including a plurality of leaflets, each leaflet having an outflow edge and a cusp edge; aligning one or more alignment features on the cusp edge of each leaflet with one or more adjacent portions of the frame; and suturing the cusp edge of each leaflet to the frame.

According to certain aspects of the disclosure, a prosthetic valve can include a radially expandable and compressible frame comprising an inflow end and an outflow end, and a leaflet assembly comprising a plurality of leaflets coupled to the frame. Each leaflet can include an outflow edge, a cusp edge opposite to the outflow edge, and a pair of commissure tabs extending from opposite sides of the leaflet and between the outflow edge and the cusp edge. Each commissure tab can be paired with an adjacent commissure tab of an adjacent leaflet to form a commissure of the leaflet assembly that is connected to the frame. The outflow edge of each leaflet can include a coaptation edge region and cut-out regions between opposite ends of the coaptation edge region and the commissure tabs. Under a forward flow of blood through the leaflet assembly, the coaptation edge regions can pivot relative to the commissure tabs at respective primary pivot axes intersecting the cut-out regions to move the leaflet assembly from a closed state to a partially opened state, after which the commissure tabs can pivot relative to each other at respective secondary pivot axes to move the leaflets from the partially opened state to a fully opened state.

Certain aspects of the disclosure concern a method including delivering a prosthetic device in a radially compressed state to a target location, and radially expanding the prosthetic device to a radially expanded state. The prosthetic device can be any one of the prosthetic valves described above.

The above method can be performed on a living animal or on a simulation, such as on a cadaver, cadaver heart, anthropomorphic ghost, simulator (for example, with body parts, heart, tissue, etc. being simulated).

In some examples, a prosthetic valve comprises one or more of the components recited in Examples 1-56 and 73-87 described in the section “Additional Examples of the Disclosed Technology” below.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 is a side view of a prosthetic heart valve, according to one example.

FIG. 2 is a side view of a frame of the prosthetic heart valve of FIG. 1.

FIG. 3 is a side view of a portion of the frame of FIG. 2, showing the portion of the frame in a straightened (non-annular) state.

FIG. 4 is a side view of an exemplary delivery apparatus configured to deliver and implant a radially expandable prosthetic valve at an implantation site.

FIG. 5 is a flattened view of a leaflet, according to one example.

FIG. 6A is a cross-sectional view of a portion of a frame and a leaflet structure showing adjacent commissure tabs of two leaflets of the type shown in FIG. 5 secured to a corresponding commissure window of the frame, according to one example.

FIG. 6B is side view of a commissure tab of the leaflet of FIG. 5 when secured to a corresponding commissure window of a frame, according to one example.

FIG. 7A is a top view of a prosthetic valve having a leaflet structure, wherein the leaflet structure is shown in a closed state, according to one example.

FIG. 7B is a top view of the prosthetic valve of FIG. 7A in which the leaflet structure is shown in a partially open state, according to one example.

FIG. 7C is a top view of the prosthetic valve of FIG. 7A in which the leaflet structure is shown in a fully open state, according to one example.

FIG. 8 depicts a portion of a leaflet for a prosthetic heart valve, according to another example.

FIG. 9A depicts a portion of a leaflet for a prosthetic heart valve, according to yet another example.

FIG. 9B depicts a commissure tab of the leaflet of FIG. 9A when coupled to a commissure window of a frame.

FIG. 10 depicts a portion of a leaflet for a prosthetic heart valve, according to a further example.

FIG. 11 is a flattened view of a leaflet, according to another example.

FIG. 11A is side view of a commissure tab of the leaflet of FIG. 11 when attached to a corresponding commissure window of a frame, according to one example.

FIG. 12 is a flattened view of a leaflet, according to a further example.

FIG. 13A depicts a portion of the leaflet of FIG. 11 being attached to the frame of FIG. 2.

FIG. 13B is an enlarged view of section A of FIG. 13A.

FIG. 14 is an enlarged view showing the alignment and attachment of the apex portion of the leaflet of FIG. 13 to a pair of struts of the frame.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language. Further, as used herein, “and/or” means “and” or “or,” as well as “and” and “or.”

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

Directions and other relative references (for example, inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

### Overview of Prosthetic Valves

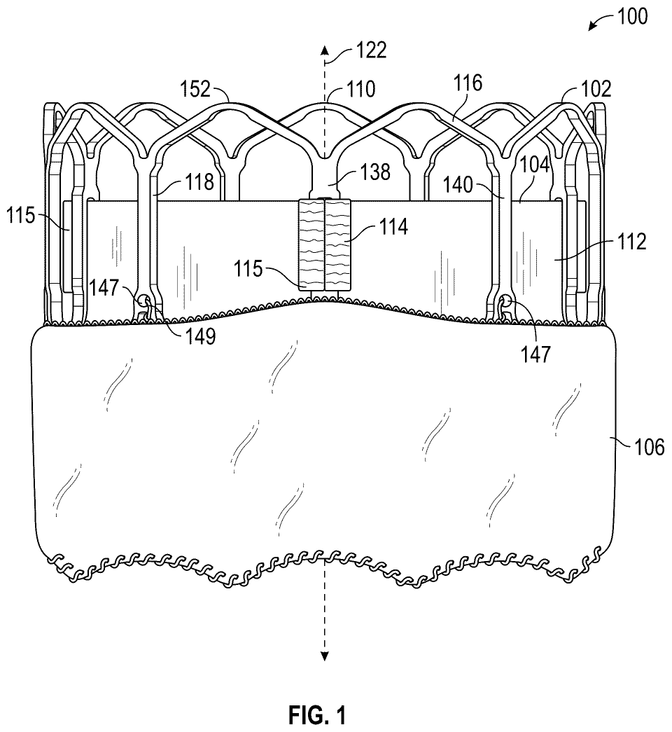

FIG. 1 shows a prosthetic heart valve 100 (or simply, “prosthetic valve”), according to one example. Any of the prosthetic valves disclosed herein can be adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic heart valve 100 can include an annular stent or frame 102, a valvular structure 104, and a perivalvular outer sealing member or outer skirt 106. The prosthetic heart valve 100 (and the frame 102) can have an inflow end 108 and an outflow end 110. The valvular structure 104 can be disposed on an interior of the frame 102 while the outer skirt 106 is disposed around an outer surface of the frame 102.

The valvular structure 104 can comprise a plurality of leaflets 112 (for example, three leaflets, as shown in FIG. 1), collectively forming a leaflet structure (also referred to as a “leaflet assembly”), which can be arranged to collapse in a tricuspid arrangement. The leaflets 112 can be secured to one another at their adjacent sides (for example, commissure tabs 115) to form commissures 114 of the valvular structure 104. For example, each leaflet 112 can comprise opposing commissure tabs 115 disposed on opposite sides of the leaflet 112 and a cusp edge portion extending between the opposing commissure tabs. The cusp edge portion of the leaflets 112 can have an undulating, curved scalloped shape, and can be secured directly to the frame 102 (for example, by sutures). However, in alternate examples, the cusp edge portion of the leaflets 112 can be secured to an inner skirt which is then secured to the frame 102. In some examples, the leaflets 112 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein. Example structures of leaflets 112 are described more fully below.

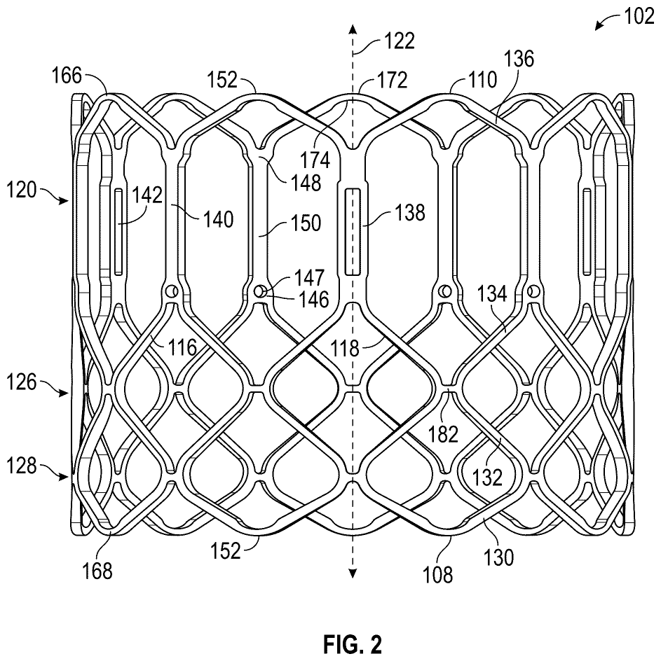

In some examples, the outer skirt 106 can be an annular skirt. In some instances, the outer skirt 106 can comprise one or more skirt portions that are connected together and/or individually connected to the frame 102. The outer skirt 106 can comprise a fabric or polymeric material, such as ePTFE, PTFE, PET, TPU, UHMWPE, PEEK, PE, etc. In some instances, instead of having a relatively straight upper edge portion, as shown in FIG. 1, the outer skirt 106 can have an undulating upper edge portion that extends along and is secured to some angled struts (for example, the angled struts 134 of FIG. 2). Examples of such outer skirts, as well as various other outer skirts, that can be used with the frame 102 can be found in U.S. provisional patent application No. 63/366,599 filed Jun. 17, 2022, which is incorporated by reference herein.

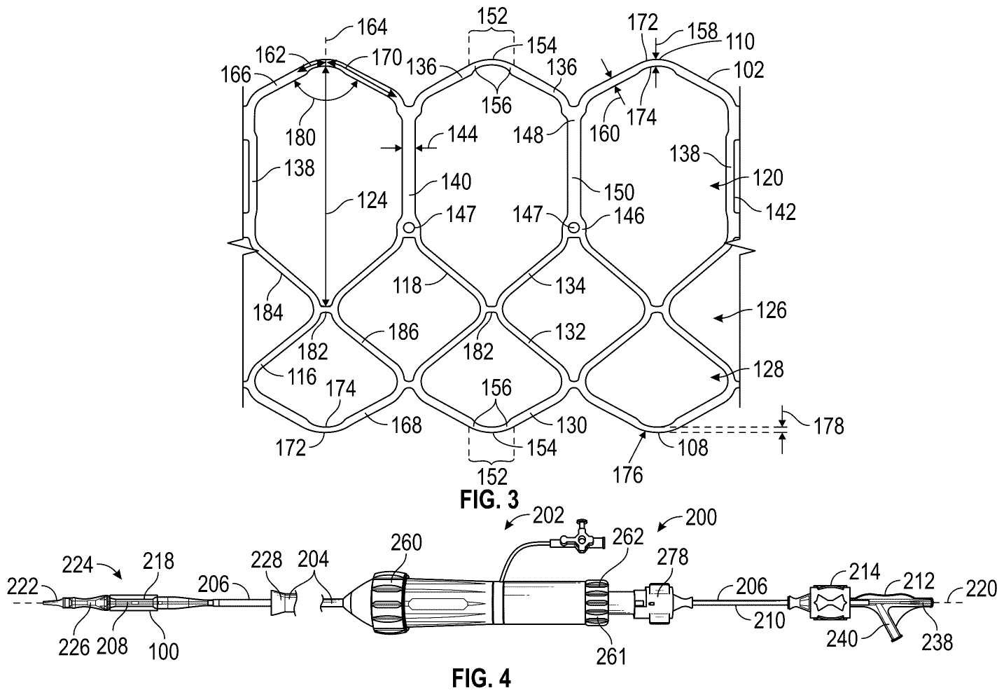

The frame 102 can be radially compressible and expandable between a radially compressed (or collapsed) configuration and a radially expanded configuration (the expanded configuration is shown in FIG. 1). The frame 102 is shown alone in FIG. 2 and a portion of the frame 102 in a straightened (non-annular) configuration is shown in FIG. 3.

The frame 102 can be made of any of various suitable plastically-expandable materials (for example, stainless steel, etc.) or self-expanding materials (for example, nickel titanium alloy (NiTi), such as nitinol). When constructed of a plastically-expandable material, the frame 102 (and thus the valve 100) can be crimped to a radially compressed state on a delivery catheter and then expanded inside a patient by an inflatable balloon or equivalent expansion mechanism. When constructed of a self-expandable material, the frame 102 (and thus the valve 100) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

As shown in FIGS. 2-3, the frame 102 can comprise a plurality of interconnected struts 116 which form multiple rows of open cells 118 between the outflow end 110 and the inflow end 108 of the frame 102. In some examples, the frame 102 can comprise three rows of cells 118 including a first (the upper row in the orientation shown in FIGS. 2-3) row 120 of cells 118 disposed at the outflow end 110, a second row 126 of cells 118 upstream of the first row 120 of cells, and a third row 128 of cells 118 at the inflow end 108 of the frame 102. The first row 120 comprises cells 118 that are elongated in an axial direction (relative to a central longitudinal axis 122 of the frame 102), as compared to cells 118 in the remaining rows of cells. For example, the cells 118 of the first row 120 of cells can have a longer axial length 124 than cells 118 in the second row 126 of cells and the third row 128 of cells.

In some examples, as shown in FIG. 2, each row of cells comprises nine cells 118. Thus, in such examples, the frame 102 can be referred to as a nine-cell frame.

In alternate examples, the frame 102 can comprise more than three rows of cells (for example, four or five) and/or more or less than nine cells per row. In some examples, the cells 118 in the first row 120 of cells may not be elongated compared to cells 118 in the remaining rows of cells of the frame 102 (for example, the second row 126 of cells and the third row 128 of cells).

The interconnected struts 116 can include a plurality of angled struts 130, 132, 134, and 136 arranged in a plurality of rows of circumferentially extending rows of angled struts, with the rows being arrayed along the length of the frame 102 between the outflow end 110 and the inflow end 108. For example, the frame 102 can comprise a first row of angled struts 130 arranged end-to-end and extending circumferentially at the inflow end 108 of the frame; a second row of circumferentially extending, angled struts 132; a third row of circumferentially extending, angled struts 134; and a fourth row of circumferentially extending, angled struts 136 at the outflow end 110 of the frame 102.

In the depicted example, the fourth row of angled struts 136 is upstream of the third row of angled struts 134, which is upstream of the second row of angled struts 132, which is upstream of the first row of angled struts 130. As described herein, a frame component (for example, a row of angled struts) is deemed to be upstream of a reference object (for example, another row of angled struts) if the frame component is closer to the inflow end 108 (or farther away from the outflow end 110) than the reference object.

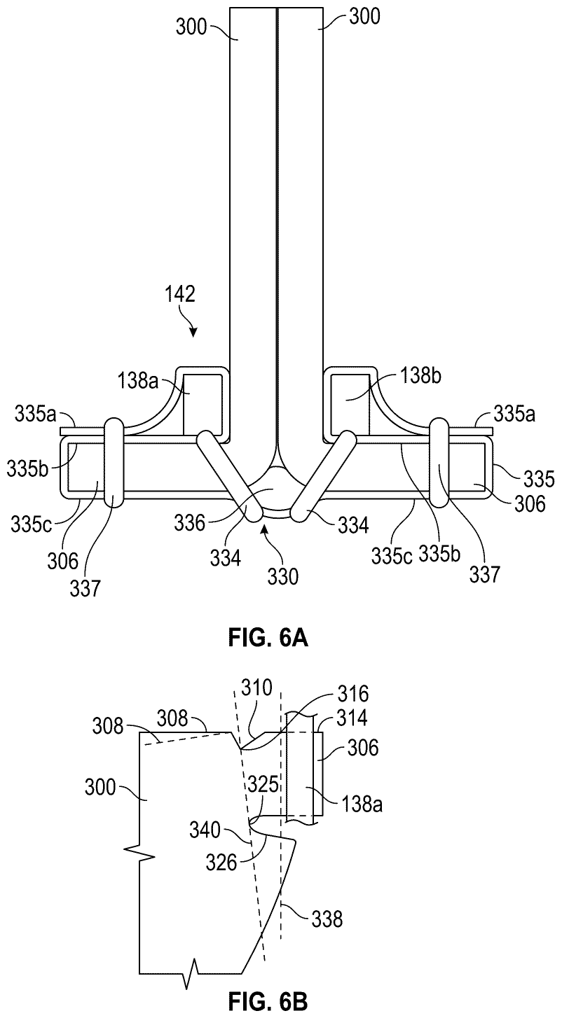

The two rows of angled struts that are closest to the outflow end 110 (for example, the fourth row of angled struts 136 and the third row of angled struts 134) can be connected by a plurality of axial (or axially extending) struts 140. Some of the axial struts 140 can define commissure windows 142 (for example, open windows extending through a thickness of the axial struts). As described herein, such axial struts with commissure windows 142 can also be referred to as axially extending window struts 138a, 138b (or simply “window struts”). Each of the windows 142 comprise a pair of window struts 138a, 138b (FIG. 6) that are be spaced apart from one another around the frame 102, in a circumferential direction. Each commissure window 142 can be adapted to receive a pair of commissure tabs of a pair of adjacent leaflets 112 arranged into a commissure (for example, commissure 114 shown in FIG. 1). In some examples, the commissure windows 142 and/or the window struts 138 defining the commissure windows 142 can be referred to as commissure features or commissure supports. Each commissure feature or support is configured to receive and/or be secured to a pair of commissure tabs of a pair of adjacent leaflets.

One or more (for example, two, as shown in FIGS. 2-3) axial struts 140 without commissure windows 142 can be positioned between, in the circumferential direction, two window struts 138. Since the frame 102 can include fewer cells per row (for example, nine) and fewer axial struts 140 between each pair of window struts 138, as compared to some more traditional prosthetic heart valves, each cell 118 can have an increased width (in the circumferential direction), thereby providing a larger opening for blood flow and/or coronary access.

In certain examples, each axial strut 140 (including each window strut 138) can extend from a location defined by the convergence of the lower ends (for example, ends arranged inward of and farthest away from the outflow end 110) of two angled struts 136 (which can also be referred to as an upper strut junction or upper elongated strut junction) to another location defined by the convergence of the upper ends (for example, ends arranged closer to the outflow end 110) of two angled struts 134 (which can also be referred to as a lower strut junction or lower elongate strut junction). Each axial strut 140 (including each window strut 138) thus forms an axial side of two adjacent cells of the first row 120 of cells 118.

In some examples, as shown in FIG. 3, each axial strut 140 can have a width 144 that is larger than a width of the angled struts 130, 132, 134, and 136. As used herein, a “width” of a strut is measured between opposing locations on opposing surfaces of the strut that extend between the radially facing inner and outer surfaces of the strut (relative to the central longitudinal axis 122 of the frame 102). A “thickness” of a strut is measured between opposing locations on the radially facing inner and outer surfaces of the strut and is perpendicular to the width of the strut. In some examples, the width 144 of the axial struts 140 is 50-200%, 75-150%, or at least 100% larger than (for example, double) the width of the angled struts of the frame 102.

By providing the axial struts 140 with a width 144 that is greater than the width of other, angled struts of the frame 102, a larger contact area is provided for when the leaflets 112 contact the wider axial struts 140 during systole, thereby distributing the stress and reducing the extent to which the leaflets 112 may fold over the axial struts 140, radially outward through the cells 118. As a result, a long-term durability of the leaflets 112 can be increased.

Since the cells 118 of the frame 102 can have a relatively large width compared to alternate prosthetic valves that have more than nine cells per row, the wider axial struts 140 can be more easily incorporated into the frame 102, without sacrificing open space for blood flow and/or coronary access.

As described above, commissure tabs 115 of adjacent leaflets 112 can be secured together to form commissures 114 (FIG. 1). Each commissure 114 of the prosthetic valve 100 comprises two commissure tabs 115 paired together, one from each of two adjacent leaflets 112, and extending through a commissure window 142 of the frame 102. Each commissure 114 can be secured to a respective window strut 138 forming commissure window 142.

The cusp edge portion (for example, scallop edge) of each leaflet 112 can be secured to the frame 102 via one or more fasteners (for example, sutures). In some examples, the cusp edge portion of each leaflet 112 can be secured directly to the struts of the frame 102 (for example, angled struts 130, 132, and 134). For example, the cusp edge portions of the leaflets 112 can be sutured to the angled struts 130, 132, and 134 that generally follow the contour of the cusp edge portions of the leaflets 112.

In some examples, the cusp edge portion of the leaflets 112 can be secured to an inner skirt and the inner skirt can then be secured directly to the frame 102.

Various methods for securing the leaflets 112 to a frame, such as the frame 102, are disclosed in U.S. provisional patent applications 63/278,922, filed Nov. 12, 2021, and 63/300,302, filed Jan. 18, 2022, both of which are incorporated by reference herein.

As shown in FIGS. 2-3, in some examples, one or more of or each of the axial struts 140 can comprise an inflow end portion 146 (for example, an end portion that is closest to the inflow end 108) and an outflow end portion 148 that are widened relative to a middle portion 150 of the axial strut 140. In some instances, the inflow end portion 146 of the axial strut 140 can comprise an aperture 147. The apertures 147 can be configured to receive fasteners (for example, sutures) for attaching soft components of the prosthetic heart valve 100 to the frame 102. For example, in some instances, the outer skirt 106 can be positioned around the outer surface of the frame 102 and an upper or outflow edge portion of the outer skirt 106 can be secured to the apertures 147 by fasteners 149 (for example, sutures), as shown in FIG. 1.

The interconnected struts 116 can also comprise horizontal struts 182 that extend between adjacent cells 118 of a row of cells of the frame 102 (FIGS. 2-3). The horizontal struts 182 can extend in a circumferential direction and also be referred to as circumferentially extending struts. The horizontal struts 182 can connect angled struts of two adjacent rows of angled struts of the frame 102 to one another. For example, each horizontal strut 182 can connect to two angled struts of one row of struts (for example, struts 134 shown in FIG. 3) and two angled struts in another, adjacent row of struts (for example, struts 132 shown in FIG. 3). As a result, an angled strut 184 extending between a window strut 138 and a horizontal strut 182 and an angled strut 186 extending between the horizontal strut 182 and another horizontal strut 182 disposed adjacent to the inflow end 108 of the frame 102 can be aligned along an angled line that can follow a scallop line of the leaflets (when the leaflets are attached to the frame 102). Thus, the horizontal struts 182 can allow the angled struts (for example, 184, 186) to follow a shape that more closely matches a shape of the scallop line of the leaflets when the frame 102 is in the radially expanded configuration (as shown in FIGS. 2-3). Additionally, the horizontal struts 182 can serve as spacers that can maintain a specified gap between the angled struts when the frame 102 is in the radially compressed configuration, thereby reducing a risk of pinching the leaflets between the struts in the radially compressed configuration.

The frame 102 can further comprise a plurality of apex regions 152 formed at the inflow end 108 and the outflow end 110, each apex region 152 extending and forming a junction between two angled struts 130 at the inflow end 108 or two angled struts 136 at the outflow end 110. As such, the apex regions 152 are spaced apart from one another, in a circumferential direction at the inflow end 108 and the outflow end 110.

In certain examples, as shown in FIG. 3, each apex region 152 can comprise an apex 154 (the highest or most outward extending, in an axial direction, point) and two thinned (or narrowed) strut portions 156, one thinned strut portion 156 extending from either side of the apex 154 to a corresponding, wider, angled strut 136 (at the outflow end 110) or angled strut 130 (at the inflow end 108). In this way, each of the apex regions 152 at the outflow end 110 can form a narrowed transition region between and relative to the two angled struts 136 extending from the corresponding apex region 152 and each of the apex regions 152 at the inflow end 108 can form a narrowed transition region between and relative to the two angled struts 130 extending from the corresponding apex region 152.

The thinned strut portions 156 of the apex regions 152 can have a width 158 that is smaller than a width 160 of the angled struts 130 or 136 (FIG. 3). In some examples, the width 158 can be a uniform width (e.g., along an entire length of the strut portion 156). In some examples, the width 158 of the thinned strut portions 156 can be from about 0.06-0.15 mm smaller than the width 160 of the angled struts 130 and/or 136.

Each of the thinned strut portions 156 of the apex regions 152 can have a length 162 in a range of 0.8-1.4 mm, 0.9-1.2 mm, 0.95-1.05 mm, or about 1.0 mm (for example, ±0.03 mm). In alternate examples, the length 162 is in a range of 0.3-0.7 mm, 0.4-0.6 mm, 0.45-0.55 mm, or about 0.5 mm (for example, ±0.03 mm). Because each outflow apex region 152 can include two thinned strut portions 156 having the same length 162, a total length of the apex region 152 can be two times the length 162.

Each apex region 152 and two corresponding angled struts 136 at the outflow end 110 can form an outflow strut 166 and each apex region 152 and two corresponding angled struts 130 at the inflow end 108 can form an inflow strut 168.

Each outflow strut 166 and inflow strut 168 can have a length that includes an apex region 152 and the two angled struts 136 or 130 (or strut portions), respectively, on either side of the apex region 152. One half the total length of each outflow strut 166 and inflow strut 168 is shown in FIG. 3 as length 170, which extends from an end of one angled strut 136 or 130 to the central longitudinal axis 164. Thus, the length of each outflow strut 166 and inflow strut 168 is two times length 170. In some examples, the length 170 for half of each inflow strut 168 can be different than the length 170 for half of each outflow strut 166.

In some instances, the length of each thinned strut portion 156 can be at least 25% of the length 170 of the corresponding half outflow strut 166 or inflow strut 168. Said another way, the length of each apex region 152 (a total length being two times the length 162) can be at least 25% of the total length (two times length 170) of the outflow strut 166 or inflow strut 168. In some examples, the length of each apex region 152 can be more than 25% of the total length of the corresponding outflow strut 166 or inflow strut 168, such as 25-35%.

In some examples, each apex region 152 can comprise a curved, axially facing outer surface 172 and an arcuate or curved, axially facing inner depression 174 which forms the thinned strut portions 156. For example, the curved inner depression 174 can depress toward the curved outer surface 172 from an inner surface of the angled strut portions 156, thereby forming the smaller width thinned strut portions 156. Thus, the curved inner depressions 174 can be formed on a cell side of the apex region 152 (for example, as opposed to the outside of the apex region 152).

In some examples, the curved outer surface 172 of each apex region 152 can form a single, continuous curve from one angled strut portion 156 on a first side of the apex region 152 to another angled strut portion 156 on an opposite, second side of the apex region 152 (for example, the curved outer surface 172 can have a constant curvature).

Each apex region 152 can have a radius of curvature 176, along the curved outer surface 172 (for example, in some instances, along an entirety or an entire length of the curved outer surface 172) (FIG. 3). In some instances, the radius of curvature 176 at the apex 154 and/or along the entire curved outer surface 172 of the apex region 152 can be greater than 1 mm. In some instances, the radius of curvature 176 can be in a range of 1-20 mm, 3-16 mm, or 8-14 mm. In some instances, the radius of curvature 176 can be greater than 10 mm. The radius of curvature 176 can be dependent on (and thus change due to changes in) the width 158 (for example, the amount of reduction in width from the angled struts 130 or 136) and the length 162 of the thinned strut portions 156.

Further, a height (an axial height) 178 of the apex regions 152, which can be defined in the axial direction from an outer surface of the two angled struts 130 or 136 to the curved outer surface 172 of the apex region 152 at the apex 154, can be the width 158 of the thinned strut portions 156 (FIG. 3). In this way, the height 178 of the apex regions 152 can be relatively small and not add much to the overall axial height of the radially expanded frame 102. Thus, the leaflets 112 secured to the frame 102 (FIG. 1) can be disposed close to the inflow end 108, thereby leaving a larger open space at the outflow end 110 of the frame 102 that is not blocked by the leaflets 112.

In some examples, each of the apex region 152 can form an angle 180 between the two angled struts 130 or 136 extending from either side of the corresponding apex region 152 (FIG. 3). In some instances, the angle 180 can be in a range of 120 (not inclusive) to 140 degrees (for example, such that the angle 180 is greater than 120 degrees and less than or equal to 140 degrees).

Additional details and examples of frames for prosthetic heart valves that include apex regions can be found in PCT Application No. PCT/US2022/025687, which is incorporated by reference herein.

### Exemplary Delivery Apparatus

FIG. 4 shows a delivery apparatus 200, according to an example, that can be used to implant an expandable prosthetic valve (for example, the prosthetic valve 100 and/or any of the other prosthetic valves described herein). In some examples, the delivery apparatus 200 can be specifically adapted for use in introducing a prosthetic valve into a heart.

The delivery apparatus 200 in the illustrated example of FIG. 4 is a balloon catheter comprising a handle 202 and a steerable, outer shaft 204 extending distally from the handle 202. The delivery apparatus 200 can further comprise an intermediate shaft 206 (which also may be referred to as a balloon shaft) that extends proximally from the handle 202 and distally from the handle 202, the portion extending distally from the handle 202 also extending coaxially through the outer shaft 204. Additionally, the delivery apparatus 200 can further comprise an inner shaft 208 extending distally from the handle 202 coaxially through the intermediate shaft 206 and the outer shaft 204 and proximally from the handle 202 coaxially through the intermediate shaft 206.

The outer shaft 204 and the intermediate shaft 206 can be configured to translate (for example, move) longitudinally, along a central longitudinal axis 220 of the delivery apparatus 200, relative to one another to facilitate delivery and positioning of a prosthetic valve at an implantation site in a patient's body.

The intermediate shaft 206 can include a proximal end portion 210 that extends proximally from a proximal end of the handle 202, to an adaptor 212. A rotatable knob 214 can be mounted on the proximal end portion 210 and can be configured to rotate the intermediate shaft 206 around the central longitudinal axis 220 and relative to the outer shaft 204.

The adaptor 212 can include a first port 238 configured to receive a guidewire therethrough and a second port 240 configured to receive fluid (for example, inflation fluid) from a fluid source. The second port 240 can be fluidly coupled to an inner lumen of the intermediate shaft 206.

The intermediate shaft 206 can further include a distal end portion that extends distally beyond a distal end of the outer shaft 204 when a distal end of the outer shaft 204 is positioned away from an inflatable balloon 218 of the delivery apparatus 200. A distal end portion of the inner shaft 208 can extend distally beyond the distal end portion of the intermediate shaft 206.

The balloon 218 can be coupled to the distal end portion of the intermediate shaft 206.

In some examples, a distal end of the balloon 218 can be coupled to a distal end of the delivery apparatus 200, such as to a nose cone 222, or to an alternate component at the distal end of the delivery apparatus 200 (for example, a distal shoulder). An intermediate portion of the balloon 218 can overlay a valve mounting portion 224 of a distal end portion of the delivery apparatus 200 and a distal end portion of the balloon 218 can overly a distal shoulder 226 of the delivery apparatus 200. The valve mounting portion 224 and the intermediate portion of the balloon 218 can be configured to receive a prosthetic valve in a radially compressed state. For example, as shown schematically in FIG. 4, a prosthetic device, such as the prosthetic valve 100, can be mounted around the balloon 218, at the valve mounting portion 224 of the delivery apparatus 200.

The balloon shoulder assembly, including the distal shoulder 226, can be configured to maintain the prosthetic valve 100 (or other medical device) at a fixed position on the balloon 218 during delivery through the patient's vasculature.

The outer shaft 204 can include a distal tip portion 228 mounted on its distal end. The outer shaft 204 and the intermediate shaft 206 can be translated axially relative to one another to position the distal tip portion 228 adjacent to a proximal end of the valve mounting portion 224, when the prosthetic valve 100 is mounted in the radially compressed state on the valve mounting portion 224 and during delivery of the prosthetic valve to the target implantation site. As such, the distal tip portion 228 can be configured to resist movement of the prosthetic valve 100 relative to the balloon 218 proximally, in the axial direction, relative to the balloon 218, when the distal tip portion 228 is arranged adjacent to a proximal side of the valve mounting portion 224.

An annular space can be defined between an outer surface of the inner shaft 208 and an inner surface of the intermediate shaft 206 and can be configured to receive fluid from a fluid source via the second port 240 of the adaptor 212. The annular space can be fluidly coupled to a fluid passageway formed between the outer surface of the distal end portion of the inner shaft 208 and an inner surface of the balloon 218. As such, fluid from the fluid source can flow to the fluid passageway from the annular space to inflate the balloon 218 and radially expand and deploy the prosthetic valve 100.

An inner lumen of the inner shaft can be configured to receive a guidewire therethrough, for navigating the distal end portion of the delivery apparatus 200 to the target implantation site.

The handle 202 can include a steering mechanism configured to adjust the curvature of the distal end portion of the delivery apparatus 200. In the illustrated example, for example, the handle 202 includes an adjustment member, such as the illustrated rotatable knob 260, which in turn can be operatively coupled to the proximal end portion of a pull wire. The pull wire can extend distally from the handle 202 through the outer shaft 204 and has a distal end portion affixed to the outer shaft 204 at or near the distal end of the outer shaft 204. Rotating the knob 260 can increase or decrease the tension in the pull wire, thereby adjusting the curvature of the distal end portion of the delivery apparatus 200. Further details on steering or flex mechanisms for the delivery apparatus can be found in U.S. Pat. No. 9,339,384, which is incorporated by reference herein.

The handle 202 can further include an adjustment mechanism 261 including an adjustment member, such as the illustrated rotatable knob 262, and an associated locking mechanism including another adjustment member, configured as a rotatable knob 278. The adjustment mechanism 261 can be configured to adjust the axial position of the intermediate shaft 206 relative to the outer shaft 204 (for example, for fine positioning at the implantation site). Further details on the delivery apparatus 200 can be found in PCT Application No. PCT/US2021/047056, which is incorporated by reference herein.

Although the delivery apparatus 200 depicted in FIG. 4 is specifically adapted to deliver a balloon expandable prosthetic valve, it is to be understood that variants of the delivery apparatus 200 can be adapted for delivery of self-expandable prosthetic valves and/or mechanically expandable prosthetic valves, as described in references incorporated above.

### Exemplary Leaflets for Prosthetic Heart Valves

Example leaflets described hereinafter are configured to prevent or minimize the contact between movable portions of the leaflets and the inner surface of the frame so as to prevent or reduce abrasion of the leaflets against the frame and improve the durability of the leaflets. Additionally, example leaflets described hereinafter are configured to reduce the risk of tissue ingrowth at the commissure region without affecting leaflet coaptation. Any of the leaflets described hereinafter can replace the leaflets 112 of the prosthetic valve 100 described above.

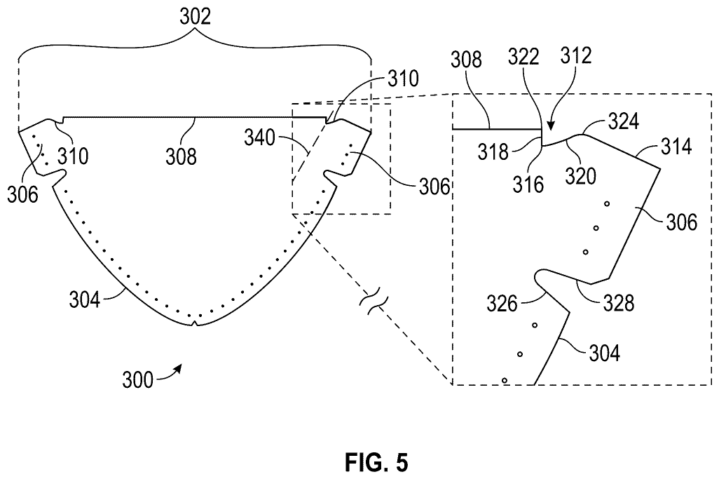

FIG. 5 shows a flattened view of a leaflet 300, according to one example. The leaflet 300 has an outflow edge 302, a cusp edge 304 opposite the outflow edge 302, and a pair of commissure tabs 306 extending from opposite sides of the leaflet 300 and between the outflow edge 302 and the cusp edge 304.

As shown, the outflow edge 302 of the leaflet 300 comprises a coaptation edge region 308 and two cut-out regions 310 (which can also be referred to as discontinuities in some examples) between opposite ends of the coaptation edge region 308 and the commissure tabs 306. In the depicted example, the cut-out regions 310 comprise V-shaped notches 312 that are adjacent to corresponding commissure tabs 306. In other words, the coaptation edge region 308 extends between the two notches 312.

Each commissure tab 306 has an outflow edge 314. The coaptation edge region 308 can be straight or substantially straight. When the leaflet 300 is flattened (prior to assembly with other leaflets and mounted to a frame), the outflow edge 314 of each commissure tab 306 can be angled relative to the coaptation edge region 308, for example, downwardly as shown in FIG. 5 (that is, toward the inflow end of the frame when mounting the leaflet to the frame).

Each notch 312 has a nadir 316, a first edge 318 extending from the nadir 316 to the coaptation edge region 308, and a second edge 320 extending from the nadir 316 to the outflow edge 314 of the corresponding commissure tab 306. In some examples, as depicted in FIG. 5, the first edge 318 is shorter than the second edge 320. In other examples, the first edge 318 can be about the same or longer than the second edge 320.

In certain examples, the first edge 318 can intersect the coaptation edge region 308 at a sharp-angled corner 322. In certain examples, the first edge 318 and the coaptation edge region 308 can form an angle of 90 degrees or greater (about 90 degrees in the example depicted in FIG. 5).

In certain examples, the second edge 320 can intersect the outflow edge 314 of the adjacent commissure tab 306 at a rounded or smoothed corner 324. In some examples, the second edge 320 and the outflow edge 314 of the adjacent commissure tab 306 can form an obtuse angle.

In certain examples, each commissure tab 306 is connected to the cusp edge 304 by a recessed region 326. For example, each commissure tab 306 can have an inflow edge 328 that is generally parallel to the outflow edge 314. The recessed region 326 can include a U-shaped or V-shaped notch formed between the cusp edge 304 and the inflow edge 328 of the commissure tab 306.

A plurality of leaflets 300 can be assembled together to form a leaflet assembly that is coupled to a frame of a prosthetic valve. For example, a prosthetic valve 100 (FIG. 1) can include a plurality of leaflets 300 instead of leaflets 112. As described above, to form the commissures of the leaflet assembly, each commissure tab 306 can be paired with an adjacent commissure tab of an adjacent leaflet 300 to form a commissure of a leaflet assembly that is connected to a frame. The leaflet assembly is movable between an open state which permits blood flow from the inflow end to the outflow end of the frame and a closed state which blocks blood fluid flow from the outflow end to the inflow end of the frame.

For example, FIG. 6A shows a commissure 330 comprising a pair of commissure tabs 306 of two adjacent leaflets that are inserted through a commissure window 142 defined between two struts 138a, 138b of the frame 102. One of the commissure tabs 306 is wrapped partially around one strut 138a on the outside of the frame 102 and the other commissure tab 306 is wrapped partially around the other strut 138b on the outside of the frame. The width of the commissure window 142 can be about the same or slightly smaller than the thickness of two commissure tabs 306 so that the pair of commissure tabs 306 are squeezed against each other inside the commissure window 142 and are tightly coupled together.

The commissure 330 can further include a reinforcing member 335, such as in the form of a piece of fabric, that assists in securing the commissure tabs 306 to the struts 138a, 138b of the frame 102. The reinforcing member 335 can comprise, for example, a piece of woven polyethylene terephthalate (PET) fabric, although other synthetic and/or natural materials can be used. The reinforcing member 335 has opposing end portion, each of which is folded to form an inner layer 335a, a first outer layer 335b, and a second outer layer 335c. Each end portion of the reinforcing member 335 is wrapped around a respective strut 138a, 138b to form a respective inner layer 335a and a respective first outer layer 335b that encloses the strut. Each second outer layer 335c extends an outer edge of a respective commissure tab 306 and along the outer surface of the commissure tab 306 where it is connected to the other second outer layer.

The reinforcing member 335 and the commissure tabs 306 can be secured to each other with one or more sutures to retain the commissure 330 on the struts 138a, 138b. For example, a pair of first suture lines 334 can be used to secure portions of the commissure tabs 306 closest to the commissure window 142. Each suture line 334 can be formed from a suture that forms stitches (for example, in-and-out stitches) that extend through a first outer layer 335b, a commissure tab 306, and a second outer layer 335c. A pair of second suture lines 337 can be used to secure the outer edge portions of the commissure tabs 306 to the reinforcing member 335. Each suture line 337 can be formed from a suture that forms stitches (for example, in-and-out stitches) that extend through an inner layer 335a, a first outer layer 335b, a commissure tab 306, and a second outer layer 335c.

In certain examples, the commissure 330 can further include a post or a wedge member 336 situated between the pair of commissure tabs 306 on the outside of the commissure window. In certain examples, the wedge member 336 can comprise a non-metallic material, such as a rope or a braided suture, such as an Ethibond suture (for example, a 2-0 Ethibond suture). The wedge member 336 can be retained against the commissure tabs 306 with the stitches of the first suture lines 334. For example, the stitches of each suture line 334 can extend through the wedge member 336 (in addition to the layers 335b, 335c of the reinforcing member 335 and the commissure tab 306).

The reinforcing member 335 and the wedge member 336 can further increase the stiffness of the commissure 330 compared to other portions of the leaflets. Other techniques and/or mechanisms can be used to form the commissure 330 and secure the commissure to a pair of posts 138a, 138b of a frame, including any of those described in U.S. Pat. No. 9,393,110 (such as the commissures disclosed in any of FIGS. 63-71 of the '110 patent) and WIPO Publication No. WO2022/056048, which are incorporated herein by reference.

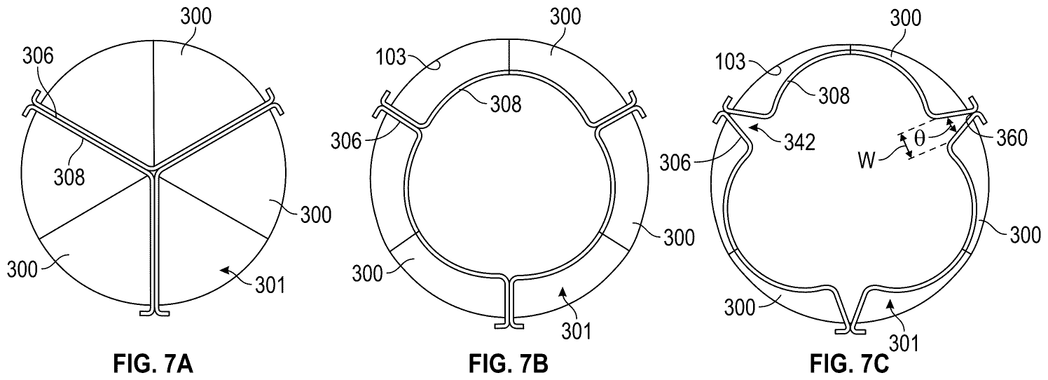

After inserting the commissure 330 into a commissure window 142 of the frame, a pivot axis 340 can be formed at the cut-out region 310 of each leaflet, extending through the nadir 316 of the corresponding notch 312. Although the pivot axis 340 depicted as being at an oblique angled relative to the coaptation edge region 308 of the flattened leaflet (see FIG. 5), in some examples, the commissure tab 306 can be pulled slightly upward when fully assembled and secured to a commissure window, which can cause the angle of the pivot axis 340 to change slightly. For example, when the leaflet assembly is in the closed state (see FIG. 7A below), the outflow edge 314 of the commissure tab 306 can be parallel or substantially parallel to the coaptation edge region 308 (and at about the same height) and perpendicular to a longitudinal axis of the prosthetic valve, as depicted in FIG. 6B. In the closed state, the pivot axis 340 can extend axially in parallel to the longitudinal axis of the prosthetic valve. When the leaflet assembly moves from the closed state to a partially open state (see FIG. 7B below), the coaptation edge region 308 can move slightly downward toward the inflow end of the frame (as indicated by the dashed line 308) and cause the pivot axis 340 to rotate accordingly. For example, the pivot axis 340 depicted in FIG. 6B is rotated in a counter-clockwise direction compared to the pivot axis depicted in FIG. 5 (and the pivot axis at the opposing tab will rotate in a clockwise direction). In some examples, when the leaflet assembly moves from the closed state to the partially open state, the pivot axis 340 can extend from the nadir 316 of the notch 312 to a nadir 325 of the recessed region 326. When the leaflet assembly moves between the partially open state and closed state, the coaptation edge region 308 of the leaflet 300 can pivot about the pivot axis 340 partially due to reduced leaflet material at the cut-out region 310 and partially due to increased stiffness at the commissure 330.

A prosthetic valve (such as a prosthetic valve 100) can include a leaflet assembly 301 comprising a plurality of leaflets 300. FIGS. 7A-7C illustrate movement of a leaflet assembly 301 comprising a plurality of leaflets 300 as blood flows through the leaflet assembly 301. The frame 102 of the prosthetic valve is omitted in FIGS. 7A-7C for purposes of illustration. In the depicted example, the leaflet assembly 301 moves from a closed state (FIG. 7A) to a partially open state (FIG. 7B) and then to a fully open state (FIG. 7C) under the forward flow of blood. As shown, in the closed state (for example, during diastole), the coaptation edge regions 308 of adjacent leaflets contact each other so as to block blood flow through the leaflet assembly. In the partially open state (for example, during early stages of systole), the coaptation edge regions 308 of adjacent leaflets bend at respective pivot axes 340 and separate from one another, thereby allowing blood flow through the leaflet assembly. In the fully open state (for example, close to the end of the systole), the coaptation edge regions 308 of adjacent leaflets can be further spaced apart, while portions of the commissure tabs 306 that are radially outward of pivot axes 340 and closer to the frame can also pivot away from each other so as to form a V-shaped gap 342 therebetween.

Note that in either the partially open state (FIG. 7B) or the fully open state (FIG. 7C), the coaptation edge regions 308 of the leaflets are spaced radially apart from the inner surface 103 of the frame 102. This is because after attaching the leaflet assembly 301 to the frame 102, the cut-out regions 310 of the leaflets extend into an interior space of the frame 102 so that the coaptation edge regions 308 are spaced radially apart from the inner surface 103 of the frame. For example, the notches 312 can space the coaptation edge regions 308 of the leaflets radially inwardly away from the inner surface 103 of the frame. Thus, as the leaflet assembly 301 moves from the closed state (FIG. 7A) to the partially open state (FIG. 7B), the leaflets 300 can pivot about the corresponding pivot axes 340, which offset or separate the coaptation edge regions 308 of the leaflets from the inner surface 103 of the frame. As a result, the likelihood of frame abrasion to the leaflets 300 can be reduced, and the durability of the leaflets 300 can be improved.

As the leaflet assembly 301 further opens, for example, from the partially open state (FIG. 7B) to the fully open state (FIG. 7C), portions of the commissure tabs 306 at each commissure can be configured to pivot relative to each other at respective pivot axes 338 (FIG. 6B) so as to form the V-shaped gap 342 therebetween. The pivot axes 338 of the commissure tabs 306 demarcates portions of the commissure tabs that are moveable and extend into the interior space of the frame and portions of the commissure tabs that are non-moveable and extend into the commissure window.

Thus, in some examples, the leaflets 300 can open according to the following sequence. First, the coaptation edge regions 308 pivot relative to the commissure tabs 306 at respective pivot axes 340 intersecting the cut-out regions 310 so that the coaptation edge regions 308 move apart from each other (for example, when moving from the closed state of FIG. 7A to the partially open state of FIG. 7B). Afterwards, the commissure tabs 306 at each commissure can pivot at the pivot axes 338 and further move apart from each other (for example, when moving from the partially open state of FIG. 7B to the fully open state of FIG. 7C). The pivot axes 340 can be referred to as primary pivot axes of the leaflets and the pivot axes 338 can be referred to as secondary pivot axes of the leaflets.

In some known prosthetic heart valves, offset pivot axes of the leaflets are accomplished by providing the commissure tabs with additional material that allows the commissure tabs to be folded form multiple layers of leaflet material that effectively offset the pivot axes of the coaptation edge regions from the inner surface of the frame. While effective to reduce leaflet abrasion, the contact between multiple layers of material at the commissures can promote tissue overgrowth or thrombus over the surfaces of the commissures, which in turn can result in relatively rigid regions of the leaflets that extend radially inward over time, and therefore may limit the effective orifice area of the valve in the open position of the leaflets. Additionally, folding of the commissure tabs in the formation of the commissures can increase the time and complexity required for assembling the valve. Advantageously, the leaflets 300 formed with notches 310 can effectively offset the pivot axes of the coaptation edge regions away from the inner surface of the frame to reduce leaflet abrasion without additional folded leaflet layers forming the commissures, which can reduce the risk of tissue overgrowth or thrombus formation at the commissures. Moreover, valve assembly is simplified because less steps are required to form the commissures.

Additionally, the gap 342 formed between the commissure tabs 306 when the leaflet assembly is in the fully open state mimic the gaps formed when native leaflets open. The gap 342 can be sufficiently large to allow a sufficient amount of blood flow or “washout” between the commissure tabs, thereby further reducing the likelihood of tissue ingrowth and thrombus or pannus formation at the commissure regions. In certain examples, the largest width (W) spanned by the gap 342 ranges between 50 μm and 350 μm, or between 100 μm and 300 μm (for example, about 100 μm), all inclusive. In certain examples, the angle (θ) formed by the gap 342 can range between 5° and 20°, or between 10° and 12° (for example, about 11°), all inclusive.

In some cases, over a period of time, a small degree of tissue formation may occur at the root 360 of the commissure between the commissure tab 306, thereby forming a small tissue bridge between the commissure tabs. The tissue bridge can function as a natural leaflet separator that can maintain a degree of separation between the commissure tabs that promotes washout between the commissure tabs. The tissue bridge can be about 100 μm and 300 μm (for example, about 100 μm) in width and can extend up to about 0.5 mm away from the commissure root 360.

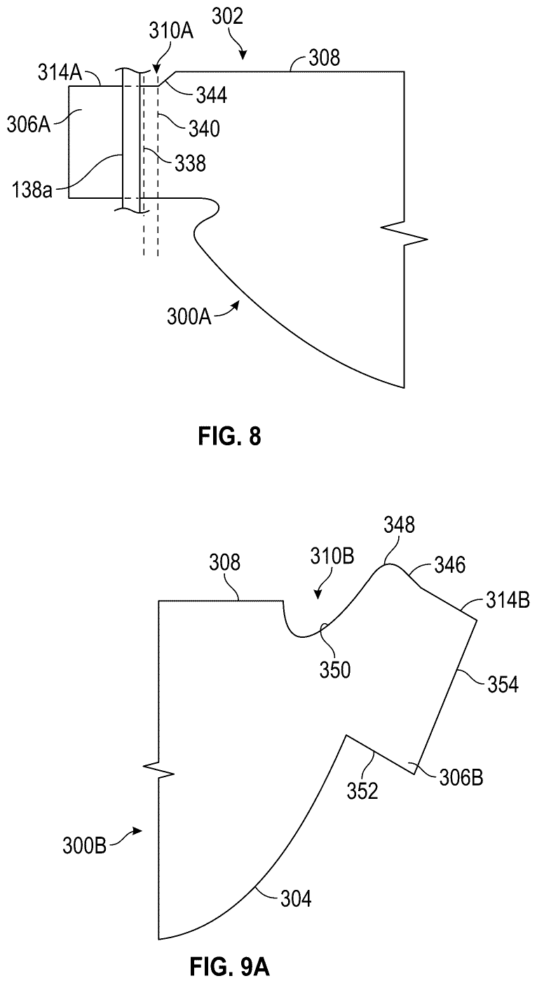

FIGS. 8-11 show alternative designs of leaflets that can also achieve the leaflet movement pattern depicted in FIGS. 7A-7C. In other words, the leaflets of FIGS. 8-11 can also prevent coaptation edge regions 308 from contacting the inner surface 103 of the frame during systole, and form sufficient gaps 342 between commissures near the end of systole to allow blood washout therein.

As one example, FIG. 8 shows a portion of the outflow edge 302 of a leaflet 300A. Similar to the example depicted in FIG. 5, the outflow edge 302 has a cut-out region 310A that separates a commissure tab 306A from the coaptation edge region 308 of the leaflet. The leaflet 300A has a commissure tab 306A on the opposite side of the leaflet and a corresponding cut-out region 310A, but are omitted in FIG. 8 for purposes of illustration. A prosthetic valve can include a leaflet assembly comprising a plurality of leaflets 300A. For example, the prosthetic valve 100 of FIG. 1 can include a plurality of leaflets 300A instead of leaflets 112. Each commissure tab 306A of each leaflet 300A can be paired with an adjacent commissure tab of an adjacent leaflet to form a commissure, which can be inserted into and coupled to a corresponding commissure window 142 of the frame 102, as previously described.

The cut-out region 310A in FIG. 8 is in the formed of a sloped transitional edge 344 that extends downwardly and radially outwardly from an end of the coaptation edge region 308 to an inner end of an outflow edge 314A of the commissure tab 306A. In some examples, as shown in FIG. 8, the sloped edge 344 can be a chamfered or straight edge. In other examples, the sloped edge 344 of the cut-out region comprises a fillet or rounded edge extending from the coaptation edge region 308 to the outflow edge 314A of the commissure tab 306A.

As shown in FIG. 8, an outflow edge 314A of each commissure tab 306A can be parallel or substantially parallel to the coaptation edge region 308 but offset axially from the coaptation edge region 308 by the sloped edge 344. In other examples, the outflow edge 314A of each commissure tab 306A can be angled relative to the corresponding coaptation edge region 308, similar to the example of FIG. 5.

Similar to the cut-out region 310, when the commissure tab 306A is coupled to a corresponding commissure window 142 of the frame 102, the cut-out regions 310A can define primary pivot axes 340 that space the coaptation edge regions 308 of the leaflets radially inwardly away from the inner surface 103 of the frame so as to reduce the risk of leaflets abrasion by the frame. Toward the end of systole, portions of the commissure tabs 306A at each commissure can further pivot relative to each other at respective secondary pivot axes 340 so as to form a V-shaped gap therebetween.

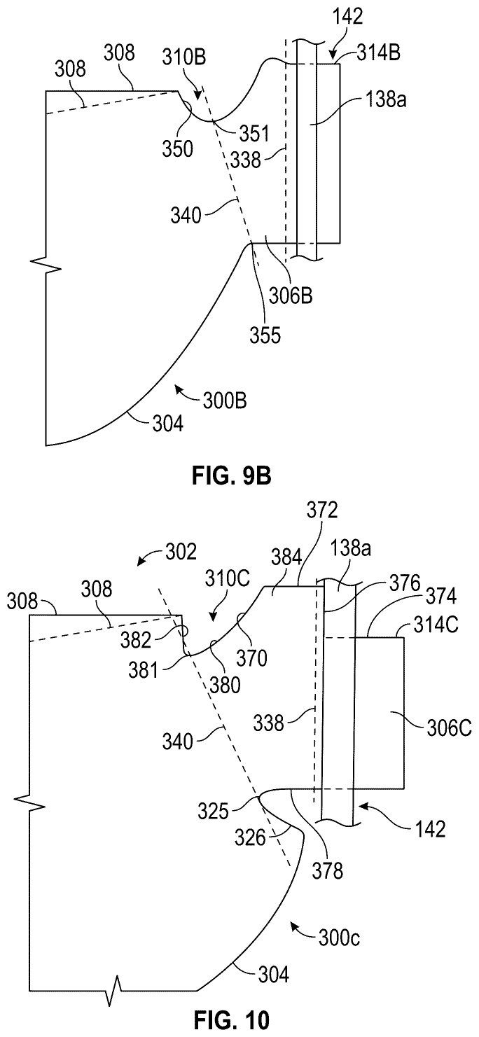

As another example, FIGS. 9A and 9B shows a portion of the outflow edge 302 of a leaflet 300B. The outflow edge 302 has a cut-out region 310B that separates a commissure tab 306B from the coaptation edge region 308 (the commissure tab on the opposite side of the leaflet and the corresponding cut-out region are omitted in FIG. 9). A prosthetic valve can include a leaflet assembly comprising a plurality of leaflets 300B. For example, the prosthetic valve 100 of FIG. 1 can include a plurality of leaflets 300B instead of leaflets 112. Each commissure tab 306B of each leaflet 300B can be paired with an adjacent commissure tab 306B of an adjacent leaflet 300B to form a commissure, which can be inserted into and coupled to a corresponding commissure window 142 of the frame 102, as previously described.

FIG. 9A shows the leaflet in a flattened state prior to forming a leaflet assembly and coupling the leaflet assembly to the frame. In the flattened state, the commissure tab 306B can be angled downwardly relative to the coaptation edge region 308, similar to the commissure tab 306 of the leaflet 300 (FIG. 5). FIG. 9B shows the commissure tab 306B of the leaflet 300B after being coupled to a commissure window of a frame, such as previously described in connection with FIG. 6A. When so coupled, the commissure tab 306B can be pulled slightly upward, which places an outflow edge 314B of the commissure tab 306B parallel or substantially parallel to the coaptation edge region 308 and perpendicular to a longitudinal axis of the prosthetic valve when the leaflet assembly is in the closed state (FIG. 7A). In other examples, the leaflet 300B can be formed such that when it is in a flattened state (before assembly), the commissure tabs 306B are parallel or substantially parallel to the coaptation edge region.

In the depicted example, the cut-out region 310B comprises a notch 350 that extends from an end of the coaptation edge region 308 to an inner end of the outflow edge 314B of the commissure tab 306B. The notch 350 can be continuously curved from the coaptation edge region 308 to the outflow edge 314B of the commissure tab 306B. The notch 350 can have a continuous concave curvature from the outflow edge 314B to the coaptation edge region 308. Additionally, the outflow edge 314B of the commissure tab 306B can include a protruding portion or “bump” 346 which extends closer to the outflow end of the frame than the remainder of the commissure tab 306B and the coaptation edge region 308. Thus, the height of the commissure tab 306B in the axial direction (the height being measured from the outflow edge 314B to an inflow edge 352 of the commissure tab) can be slightly greater near the notch 350 than it is closer an outer side edge 354 near the frame 102. In this manner, the height of the commissure tab 306B can be tapered moving in a direction from the notch 350 to the outer side edge 354. The protruding portion 346 can have a rounded or smooth edge 348 extending from the notch 350 to the outflow edge 314B of the commissure tab. As depicted in FIG. 9B, when the commissure tab 306B of the leaflet 300B is coupled to a commissure window of a frame, the outflow edge 314B of the commissure tab 306B can be higher than the coaptation edge region 308.

The notch 350 defines a primary pivot axis 340 spaced inwardly from the frame about which the coaptation edge region 308 can pivot relative to an adjacent coaptation edge region of an adjacent leaflet when the leaflets move from a closed state (FIG. 7A) to a partially open state (FIG. 7B). The primary pivot axis 340 can extend through a nadir or lowest point 351 of the notch 350. In some examples, as depicted in FIG. 9B, when the leaflet assembly moves from the closed state to the partially open state, the coaptation edge region 308 can move slightly downward toward the inflow end of the frame (as indicated by the dashed line 308), and the primary pivot axis 340 can extend from the nadir 351 of the notch 350 to a corner 355 where the inflow edge 352 of the commissure tab 306B joins the cusp edge 304. In some examples, the inflow edge 352 of the commissure tab 306B can be connected to the cusp edge 304 by a recessed region similar to 326 of FIG. 5. In such circumstances, the primary pivot axis 340 can extend from the nadir 351 of the notch 350 to a nadir of the recessed region similar to the example depicted in FIG. 6B.

A secondary pivot axis 338 for the commissure tab 306B can be defined closer to the frame. A pair of commissure tabs 306B of a commissure therefore can pivot relative to each other at respective pivot axes 338 when the leaflets move from the partially open state (FIG. 7B) to a fully open state (FIG. 7C).

When attaching the leaflet 300B to the frame, at least a portion of the protruding portion 346 is inserted into the corresponding commissure window 142 of the frame 102. As a result, more leaflet material can be compressed within the commissure window 142, thereby further increasing the stiffness of the commissure. As described above, the increased stiffness of the commissures can further contribute to distancing the coaptation edge regions 308 of the leaflets from the inner surface 103 of the frame so as to reduce the risk of leaflet abrasion by the frame. Toward the end of systole, portions of the commissure tabs 306B at each commissure can further pivot at pivot axes 338 relative to each other so as to form a V-shaped gap therebetween.

As yet another example, FIG. 10 shows a portion of the outflow edge 302 of a leaflet 300C. The outflow edge 302 has a cut-out region 310C that separates a commissure tab 306C from the coaptation edge region 308 (the commissure tab on the opposite side of the leaflet and the corresponding cut-out region are omitted in FIG. 10). A prosthetic valve can include a leaflet assembly comprising a plurality of leaflets 300C. For example, the prosthetic valve 100 of FIG. 1 can include a plurality of leaflets 300C instead of leaflets 112. Each commissure tab 306C of each leaflet 300C can be paired with an adjacent commissure tab 306C of an adjacent leaflet 300C to form a commissure, which can be inserted into and coupled to a corresponding commissure window 142 of the frame 102.

In the depicted example, the cut-out region 310C comprises a notch 370 extending from an end of the coaptation edge region 308 to an inner end of an outflow edge 314C of the commissure tab. Additionally, the commissure tab 306C includes a protruding portion 384 which extends closer to the outflow end of the frame than the coaptation edge region 308 of the corresponding leaflet.

In the example depicted in FIG. 10, the outflow edge 314C of the commissure tab 306C has a stepped configuration defining an inner lateral edge 372, an outer lateral edge 374, and an axially extending side edge 376 extending from the inner lateral edge 372 to the outer lateral edge 374. The side edge 376 can be oriented perpendicular to the lateral edges 372, 374. The commissure tab 306C therefore has an inner portion (which includes the protruding portion 384) defined between an inflow edge 378 of the commissure tab and the inner lateral edge 372 and an outer portion defined between the inflow edge 378 and the outer lateral edge 374. The commissure tab 306C has a first height along the inner portion (measured from the inflow edge 378 to the inner lateral edge 372) that is greater than a second height along the outer portion (measured from the inflow edge 378 to the outer lateral edge 374). The coaptation edge region 308 can be offset axially from the inner lateral edge 372 toward the inflow end of the leaflet and the prosthetic valve.

The notch 370 can have a first edge 380 that extends inwardly and downwardly from the inner lateral edge 372 to a nadir 381 of the notch and a second edge 382 that extends from the nadir 381 to an end of the coaptation edge region 308. The notch 370 can have a similar shape to the notch 312 of FIG. 5 except that the first edge 380 can have a concave curvature. The second edge 382 can be straight and can be oriented perpendicular to the coaptation edge region 308 as shown in FIG. 10. In other examples, the second edge 382 can be curved (for example, the second edge can have a concave curvature) and/or can extend at an obtuse or acute angled relative to the coaptation edge region 308.

As shown in FIG. 10, when the leaflet 300C is in a flattened state (prior to assembly to a frame), the commissure tab 306C and the lateral edges 372, 374 of the outflow edge 314C can be parallel or substantially parallel to the corresponding coaptation edge region 308. In other examples, the commissure tab 306C can be angled relative to the coaptation edge region 308, similar to the example of FIG. 5. For example, one or both of the lateral edges 372, 374 can extend at an oblique angle relative to the coaptation edge region 308.

When the leaflet 300C is attached to the frame, the outer portion of the commissure tab 306C (the portion defined by inflow edge 378 and the outer lateral edge 374) can extend through a corresponding commissure window 142 of the frame, and the side edge 376 can abut or can be juxtaposed to an inner surface of a strut 138a of the corresponding commissure window 142.

The notch 370 defines a primary pivot axis 340 spaced inwardly from the frame about which the coaptation edge region 308 can pivot relative to an adjacent coaptation edge region of an adjacent leaflet when the leaflets move from a closed state (FIG. 7A) to a partially open state (FIG. 7B). The primary pivot axis 340 can extend through the nadir or lowest point of the notch 370. In some examples, as depicted in FIG. 10, when the leaflet assembly moves from the closed state to the partially open state, the midportion of the coaptation edge region 308 can move slightly downward toward the inflow end of the frame (as indicated by the dashed line 308), and the primary pivot axis 340 can extend from the nadir 381 of the notch 370 to the nadir 325 of the recessed region 326 which connects the inflow edge 378 of the commissure tab 306C to the cusp edge 304. A secondary pivot axis 338 for the commissure tab 306C is defined closer to the frame, for example, along or immediately adjacent the side edge 376. A pair of commissure tabs 306C of a commissure therefore can pivot relative to each other at respective pivot axes 338 when the leaflets move from the partially open state (FIG. 7B) to a fully open state (FIG. 7C).

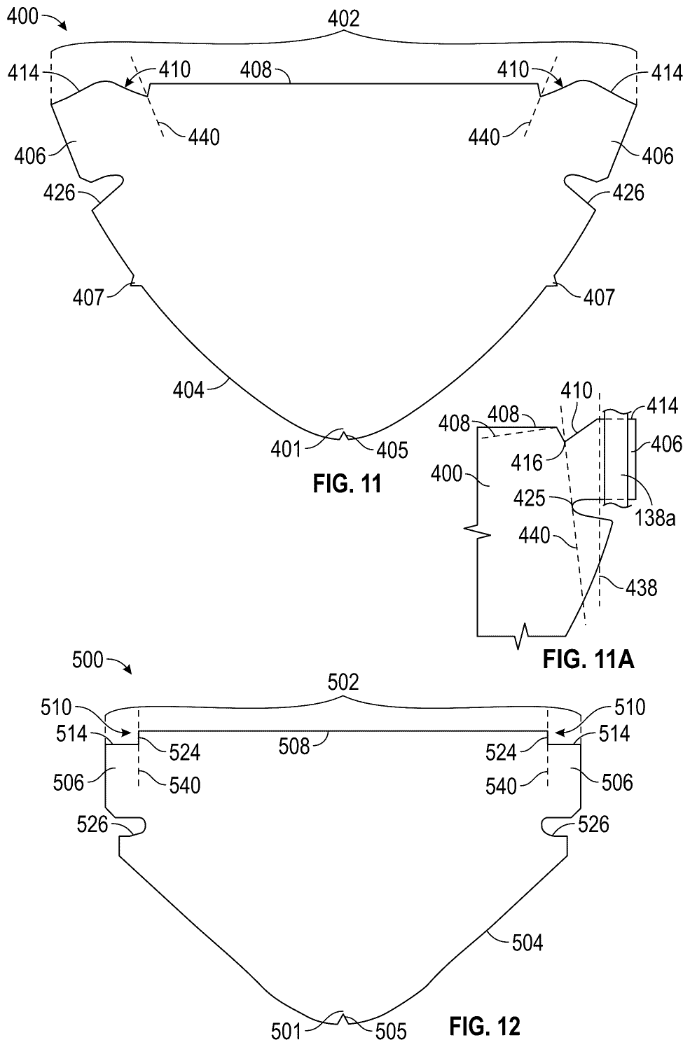

FIG. 11 shows a flattened view of a leaflet 400, according to one example. A plurality of leaflets 400 can be assembled together to form a leaflet assembly that is coupled to a frame of a prosthetic valve. For example, a prosthetic valve 100 (FIG. 1) can include a plurality of leaflets 400 instead of leaflets 112.

Similar to the leaflet 300 depicted in FIG. 5, the leaflet 400 has an outflow edge 402, a cusp edge 404 opposite the outflow edge 402, and a pair of commissure tabs 406 extending from opposite sides of the leaflet 400 and between the outflow edge 402 and the cusp edge 404. The outflow edge 402 comprises a coaptation edge region 408 and two cut-out regions 410 between opposite ends of the coaptation edge region 408 and the commissure tabs 406. An outflow edge 414 of each commissure tab 406 can be angled relative to the coaptation edge region 408. Each commissure tab 406 can be connected to the cusp edge 404 by a recessed region 426.

FIG. 11A shows the commissure tab 406 of the leaflet 400 after being coupled to a commissure window of a frame, such as previously described in connection with FIG. 6A. When so coupled, the commissure tab 306B can be pulled slightly upward, which places the outflow edge 414 of the commissure tab 406 parallel or substantially parallel to (and slightly higher than) the coaptation edge region 408 and perpendicular to a longitudinal axis of the prosthetic valve when the leaflet assembly is in the closed state (FIG. 7A). The outflow edge 414 of the commissure tab 406 can be slightly higher than the coaptation edge region 408; that is, the outflow edge 414 can extend further downstream than the coaptation edge region 408.

The cut-out regions 410 can define primary pivot axes 440 (similar to the pivot axis 340 of FIG. 5) that space the coaptation edge regions 408 of the leaflets radially inwardly away from the inner surface of the frame so as to reduce the risk of leaflets abrasion by the frame. Similar to the example depicted in FIG. 5, the primary pivot axis 440 can extend through a nadir 416 of a notch at the cut-out region 410. In some examples, as depicted in FIG. 11A, when the leaflet assembly moves from the closed state (FIG. 7A) to the partially open state (FIG. 7B), the coaptation edge region 408 can move slightly downward toward the inflow end of the frame (as indicated by the dashed line 408), and the primary pivot axis 440 can extend from the nadir 416 of the notch at the cut-out region 410 to a nadir 425 of a recessed region which connects an inflow edge of the commissure tab 406 to the cusp edge 404.

Additionally, as shown in FIG. 11A, when attaching to the frame, secondary pivot axes 438 for the commissure tabs 406 can be formed closer to the frame (similar to the pivot axis 338 of FIG. 6B). A pair of commissure tabs 406 of a commissure therefore can pivot relative to each other at respective pivot axes 438 when the leaflets move from the partially open state (FIG. 7B) to a fully open state (FIG. 7C).

The leaflet 400 is generally the same as leaflet 300 of FIG. 5. One difference is that the leaflet 400 has one or more alignment features in the form of two small protrusions 407 extending outwardly from each side of the cusp edge 404. In the depicted example, each protrusion 407 is V-shaped. In other examples, the protrusions 407 can have other shapes (for example, U-shape, semi-circle, etc.). Additionally, an apex region 401 of the leaflet 400 can have a small notch 405 (FIG. 5 also shows a small notch located in the apex region of the leaflet 300). In the depicted example, the notch 405 is V-shaped. In other examples, the notch 405 can have other shapes (for example, U-shape, semi-circle, etc.). The two protrusions 407 can be located symmetric about the notch 405. The distance between each protrusion 407 and the notch 405 along the cusp edge can be predefined so that when attaching the leaflet 400 to a frame, the protrusions 407 can align with corresponding struts of the frame to facilitate valve assembly, as described below.

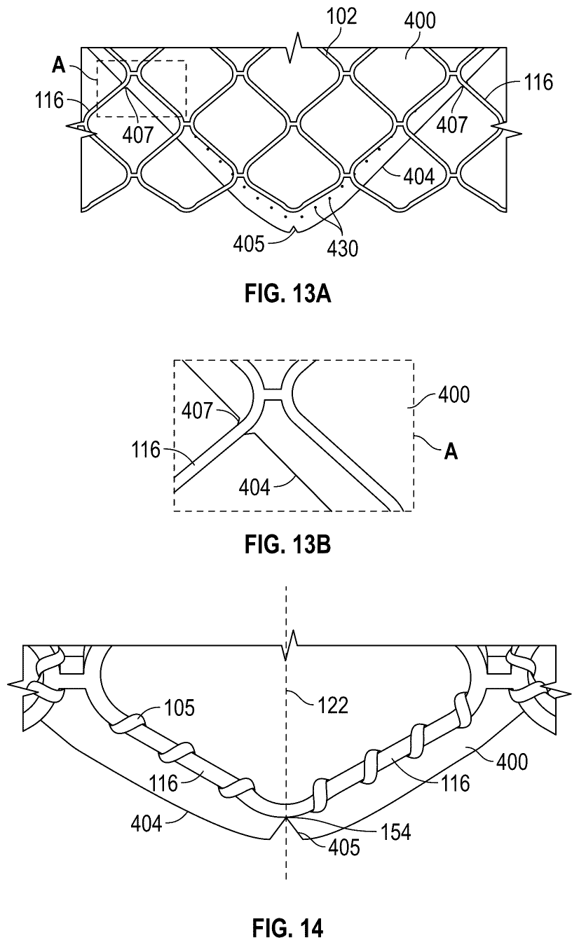

Symmetry of leaflet assembly can be crucial for valve performance. As shown in FIGS. 13A, 13B, and 14, the protrusions 407 and/or the notch 405 can be used to guide proper leaflet alignment and ensure symmetry of leaflet assembly when attaching a plurality of leaflets 400 to a frame (for example, the frame 102). As depicted in FIGS. 13A-13B, when attaching the leaflet 400 to the frame 102, each protrusion 407 can be aligned with the longitudinal axis of a corresponding angled strut 116 (for example, one of the angled struts 134 in the third row of angled struts 116). Additionally, as depicted in FIG. 14, when attaching the leaflet 400 to the frame 102, the notch 405 at the apex region of the leaflet 400 can be aligned with an apex 154 at the inflow end 108 of the frame 102 such that the tip of the notch 405 can point to the center of the apex. When it is confirmed that each protrusion 407 is properly aligned with the longitudinal axis of an adjacent angled strut 116 and the notch 405 is centrally aligned with the apex 154 at the inflow end 108, the leaflet 400 can be tightly stitched to the struts 116 of the frame 102, for example, by one or more sutures 105. The one or more sutures 105 can form whip stitches that extend through the cusp edge 404 and around adjacent struts 116. The leaflet 400 can have pre-formed apertures 430 spaced along the cusp edge 404 for receiving the sutures 105. Alternatively, the cusp edge of each leaflet can be indirectly sutured to the frame 102 by, for example, threading the whip stitches of a suture 105 through other stiches formed along the cusp edges 404 of each leaflet 400, such as disclosed in PCT Application No. PCT/US2022/049666, filed Nov. 11, 2022, which is incorporated by reference herein. Further details for forming a valve assembly and assembling it to a frame of a prosthetic heart are disclosed in PCT Application No. PCT/US2022/049666, and any such details or features disclosed in the prior application can be implemented in any of the prosthetic heart valves disclosed herein.

In some examples, instead of having protrusions 407 and the notches 405, the leaflets 400 can have visible markers (for example, colored markers) at the corresponding locations to assist proper alignment and symmetrical attachment of the leaflets 400 to the frame. In some examples, the notch 405 can be replaced with a protrusion 407 and/or one or both of the protrusions 407 can be replaced with a notch 405. In some examples, the cusp edge 404 can have more than two protrusions 407 (or notches 405) for aligning the cusp edge with struts 116 in multiple rows of struts.

FIG. 12 shows a flattened view of a leaflet 500, according to one example. A plurality of leaflets 500 can be assembled together to form a leaflet assembly that is coupled to a frame of a prosthetic valve. For example, a prosthetic valve 100 (FIG. 1) can include a plurality of leaflets 500 instead of leaflets 112. In some examples, the leaflet 500 can be smaller than the leaflets 300, 300A, 300B, 300C, and 400 for use in relatively smaller prosthetic valves. For example, a 20-mm or 23-mm prosthetic valve can include a leaflet assembly formed from leaflets 500, while a relatively larger prosthetic valve, such as a 29-mm prosthetic valve can include a leaflet assembly formed from leaflets 300, 300A, 300B, 300C, or 400. However, it should be noted that the leaflets 300, 300A, 300B, 300C, 400, and 500 are not limited to use in certain size valves. Thus, in some examples, it may be desirable to use leaflet 500 in a larger valve or any of leaflets 300, 300A, 300B, 300C, and 400 in a smaller valve.

Similar to the leaflet 300A of FIG. 8, the leaflet 400 has an outflow edge 502, a cusp edge 504 opposite the outflow edge 402, and a pair of commissure tabs 506 extending from opposite sides of the leaflet 500 and between the outflow edge 502 and the cusp edge 504. The outflow edge 502 comprises a coaptation edge region 508 and two cut-out regions 510 between opposite ends of the coaptation edge region 508 and the commissure tabs 506. Each commissure tab 506 can be connected to the cusp edge 504 by a recessed region 526. Similar to the leaflet 400 of FIG. 11, an apex region 501 of the leaflet 500 can have a small notch 505 used for guide symmetrical alignment of the leaflet 500 to a frame. Although not shown, in some examples, the leaflet 500 can also have additional alignment features, such as small protrusions (similar to the protrusions 407 of FIG. 11) or markers on the sides of the cusp edge 504 to assist proper alignment of the leaflet 500 with angled struts of the frame.

The cut-out region 510 in FIG. 12 can be formed in a stepped configuration. Specifically, each cut-out region 510 comprises an axially extending side edge 524 connecting (and being perpendicular to) the coaptation edge region 508 and an outflow edge 514 of the corresponding commissure tab 506.

When attaching to the frame, the cut-out regions 510 can define pivot axes 540 along the side edges 524. The coaptation edge region 508 can pivot at the pivot axes 540 from the closed state to the fully opened state of the leaflet assembly. It has been observed that leaflet abrasion by the frame is less of a concern for smaller prosthetic valves than larger prosthetic valves. Thus, even if the coaptation edge regions 508 of the leaflets 500 may contact the frame, the risk of leaflets abrasion can be small due to the smaller size of the leaflets 500. In some examples, to further reduce the risk of leaflet abrasion, the cut-out regions 510 can be configured to have sloped transitional edges similar to 344 of FIG. 8 so as to space the coaptation edge regions 508 of the leaflets radially inwardly away from the inner surface of the frame.

### Exemplary Delivery Techniques

For implanting a prosthetic valve within the native aortic valve via a transfemoral delivery approach, the prosthetic valve can be mounted in a radially compressed state along the distal end portion of a delivery apparatus. The prosthetic valve and the distal end portion of the delivery apparatus can be inserted into a femoral artery and then advanced into and through the descending aorta, around the aortic arch, and through the ascending aorta. The prosthetic valve can be positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) can be introduced into the left ventricle through a surgical opening in the chest and the apex of the heart and the prosthetic valve can be positioned within the native aortic valve. Alternatively, in a transaortic procedure, a prosthetic valve (on the distal end portion of the delivery apparatus) can be introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve can be mounted in a radially compressed state along the distal end portion of a delivery apparatus. The prosthetic valve and the distal end portion of the delivery apparatus can be inserted into a femoral vein and then advanced into and through the inferior vena cava, into the right atrium, across the atrial septum (for example, through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) can be introduced into the left ventricle through a surgical opening in the chest and the apex of the heart and the prosthetic valve can be positioned within the native mitral valve.

For implanting a prosthetic valve within the native tricuspid valve, the prosthetic valve can be mounted in a radially compressed state along the distal end portion of a delivery apparatus. The prosthetic valve and the distal end portion of the delivery apparatus can be inserted into a femoral vein and then advanced into and through the inferior vena cava, and into the right atrium, and the prosthetic valve can be positioned within the native tricuspid valve. A similar approach can be used for implanting the prosthetic valve within the native pulmonary valve or the pulmonary artery, except that the prosthetic valve can be advanced through the native tricuspid valve into the right ventricle and toward the pulmonary valve/pulmonary artery.

Another delivery approach is a trans-atrial approach whereby a prosthetic valve (on the distal end portion of the delivery apparatus) can be inserted through an incision in the chest and an incision made through an atrial wall (of the right or left atrium) for accessing any of the native heart valves. Atrial delivery can also be made intravascularly, such as from a pulmonary vein. Still another delivery approach is a trans-ventricular approach whereby a prosthetic valve (on the distal end portion of the delivery apparatus) can be inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Exemplary Materials and Expansion Mechanisms for Prosthetic Valves

For any of the prosthetic valves described herein, the frame can be made of any of various suitable plastically-expandable materials. When constructed of a plastically-expandable material, the frame (and thus the prosthetic valve) can be crimped to a radially collapsed configuration on a delivery catheter or apparatus and then expanded inside a patient by an inflatable balloon or equivalent expansion mechanism. In certain examples, during delivery to the implantation site, the prosthetic valve can be placed inside of a delivery capsule or sheath to protect against the prosthetic valve contacting the patient's vasculature, such as when the prosthetic valve is advanced through a femoral artery. The capsule can also retain the prosthetic valve in a radially compressed state having a slightly smaller diameter and crimp profile than may be otherwise possible without a capsule by preventing any recoil (expansion) of the frame once it is crimped onto the delivery apparatus.

Suitable plastically-expandable materials that can be used to form the frame include, without limitation, stainless steel, a biocompatible, high-strength alloys (for example, a cobalt-chromium or a nickel-cobalt-chromium alloys), polymers, or combinations thereof. In particular examples, frame is made of a nickel-cobalt-chromium-molybdenum alloy, such as MP35N® alloy (SPS Technologies, Jenkintown, Pennsylvania), which is equivalent to UNS R30035 alloy (covered by ASTM F562-02). MP35N® alloy/UNS R30035 alloy comprises 35% nickel, 35% cobalt, 20% chromium, and 10% molybdenum, by weight.

Additional details on balloon expandable prosthetic valves can be found in U.S. Pat. No. 9,393,110, and U.S. Provisional Application Nos. 63/178,416, filed Apr. 22, 2021, 63/194,830, filed May 28, 2021, and 63/279,096, filed Nov. 13, 2021, all of which are incorporated by reference herein.

Any of the prosthetic valves described herein can be self-expandable. For example, the frame of the prosthetic valve can comprise a shape-memory material (for example, Nitinol). When the prosthetic valve is self-expandable, the frame (and thus the prosthetic valve) can be crimped to a radially compressed configuration and restrained in the compressed configuration by insertion into a sheath or equivalent mechanism of a delivery catheter. Once inside the body at the desired implantation site, the prosthetic valve can be deployed or released from the delivery sheath, which allows the prosthetic valve to expand to its functional size. In some examples, the frame (and therefore the prosthetic valve) can partially self-expand from the radially compressed configuration to a partially radially expanded configuration. The frame (and therefore the prosthetic valve) can be further radially expanded from the partially expanded configuration to a further radially expanded configuration via one or more actuation assemblies (for example, an inflatable balloon and/or one or more mechanical actuators) of the delivery apparatus.

Additional details regarding exemplary self-expandable prosthetic valves and the related delivery apparatus/catheters/systems are described in U.S. Pat. Nos. 8,652,202, 9,155,619, and 9,867,700, all of which are incorporated herein by reference.

Additionally, and/or alternatively, any of the prosthetic valves described herein can be mechanically expandable. For example, the struts of the frame can be pivotably coupled to one another at one or more pivot joints along the length of each strut. An axial force applied to the frame (for example, pressing the inflow end and the outflow end of the frame toward each other or pulling the inflow end and the outflow end of the frame away from each other) can cause the prosthetic valve to radially expand or compress. The axial force can be generated by actuating one or more mechanical actuators of the delivery apparatus that are operatively coupled to the frame.

Additional details regarding exemplary mechanically-expandable prosthetic valves and the related delivery apparatus/catheters/systems are described in U.S. Patent Application Publication Nos. 2018/0153689, 2018/0311039, 2019/0060057, and PCT Patent Application Publication No. WO/2021/188476, all of which are incorporated by reference herein.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic valve, comprising: a radially expandable and compressible frame comprising an inflow end and an outflow end; and a leaflet assembly comprising a plurality of leaflets coupled to the frame, wherein the leaflet assembly is movable between an open state which permits blood flow from the inflow end to the outflow end and a closed state which blocks blood fluid flow from the outflow end to the inflow end, wherein each leaflet comprises an outflow edge, a cusp edge opposite to the outflow edge, and a pair of commissure tabs extending from opposite sides of the leaflet and between the outflow edge and the cusp edge, wherein each commissure tab is paired with an adjacent commissure tab of an adjacent leaflet to form a commissure of the leaflet assembly that is connected to the frame, wherein the outflow edge of each leaflet comprises a coaptation edge region and cut-out regions between opposite ends of the coaptation edge region and the commissure tabs, wherein when the leaflet assembly moves between the open and closed states, the coaptation edge regions pivot relative to the commissure tabs at respective pivot axes intersecting the cut-out regions.

Example 2. The prosthetic valve of any example herein, particularly example 1, wherein the cut-out regions comprise notches formed in the outflow edge of each leaflet.

Example 3. The prosthetic valve of any example herein, particularly example 2, wherein each pivot axis extends through a nadir of a corresponding notch.

Example 4. The prosthetic valve of any example herein, particularly example 3, wherein each notch comprises a first edge extending between the nadir and a corresponding coaptation edge region, wherein the first edge and the corresponding coaptation edge region form an angle of 90 degrees or greater.

Example 5. The prosthetic valve of any example herein, particularly example 4, wherein each notch comprises a second edge extending between the nadir and an outflow edge of a corresponding commissure tab, wherein the second edge and the outflow edge of the corresponding commissure tab form an obtuse angle.

Example 6. The prosthetic valve of any example herein, particularly example 5, wherein when a leaflet is flattened, the outflow edge of each commissure tab of the leaflet is angled relative to the corresponding coaptation edge region.

Example 7. The prosthetic valve of any example herein, particularly example 1, wherein each commissure tab comprises a protruding portion which extends closer to the outflow end than the coaptation edge region of the corresponding leaflet.

Example 8. The prosthetic valve of any example herein, particularly example 7, wherein the protruding portion has a rounded edge connecting an outflow edge of the commissure tab to a corresponding cut-out region.

Example 9. The prosthetic valve of any example herein, particularly example 8, wherein at least a portion of the protruding portion is inserted into a corresponding commissure window of the frame.

Example 10. The prosthetic valve of any example herein, particularly example 7, wherein the protruding portion has a side edge connected to and oriented perpendicular to an outflow edge of the commissure tab.

Example 11. The prosthetic valve of any example herein, particularly example 10, wherein the outflow edge of the commissure tab extends through a corresponding commissure window of the frame, wherein the side edge abuts an inner surface of the corresponding commissure window.

Example 12. The prosthetic valve of any example herein, particularly example 1, wherein the cut-out regions comprise chamfered edges formed in the outflow edge of each leaflet.

Example 13. The prosthetic valve of any example herein, particularly example 1, wherein the cut-out regions comprise rounded edges formed in the outflow edge of each leaflet.

Example 14. The prosthetic valve of any example herein, particularly any one of examples 12-13, wherein each commissure tab extends through a corresponding commissure window of the frame, and wherein the cut-out regions space the coaptation edge regions of the leaflets radially inwardly away from an inner surface of the frame.

Example 15. The prosthetic valve of any example herein, particularly any one of examples 12-14, wherein when a leaflet is flattened, an outflow edge of each commissure tab of the leaflet is parallel to the corresponding coaptation edge region.

Example 16. The prosthetic valve of any example herein, particularly any one of examples 1-15, wherein each commissure tab is connected to the corresponding cusp edge by a recessed region.

Example 17. The prosthetic valve of any example herein, particularly any one of examples 1-16, wherein each commissure of the leaflet assembly further comprises a wedge member between the corresponding pair of commissure tabs on outside of the frame.

Example 18. The prosthetic valve of any example herein, particularly any one of examples 1-17, wherein the coaptation edge regions are straight or substantially straight.

Example 19. The prosthetic valve of any example herein, particularly any one of examples 1-18, wherein each leaflet comprises exactly two commissure tabs.

Example 20. The prosthetic valve of any example herein, particularly any one of examples 1-19, wherein portions of the commissure tabs at each commissure are configured to pivot relative to each other when the leaflet assembly moves between the open and closed states.

Example 21. The prosthetic valve of any example herein, particularly example 20, wherein when the leaflet assembly moves from the closed state to the open state, the coaptation edge regions move apart from each other, after which the commissure tabs at each commissure move apart from each other.

Example 22. A prosthetic valve, comprising: a radially expandable and compressible frame comprising an inflow end and an outflow end; and a leaflet assembly comprising a plurality of leaflets coupled to the frame, wherein each leaflet comprises an outflow edge, a cusp edge opposite to the outflow edge, and a pair of commissure tabs extending from opposite sides of the leaflet and between the outflow edge and the cusp edge, wherein the outflow edge of each leaflet is formed with a notch adjacent each commissure tab.

Example 23. The prosthetic valve of any example herein, particularly example 22, wherein the outflow edge of each leaflet defines a coaptation edge region extending between the notches.

Example 24. The prosthetic valve of any example herein, particularly example 23, wherein each notch has a V-shape defined by a first edge and a second edge.

Example 25. The prosthetic valve of any example herein, particularly example 24, wherein the first edge intersects a corresponding coaptation edge region at a sharp-angled corner.

Example 26. The prosthetic valve of any example herein, particularly any one of examples 24-25, wherein the second edge intersects an outflow edge of an adjacent commissure tab at a rounded corner.

Example 27. The prosthetic valve of any example herein, particularly any one of examples 24-26, wherein the first edge is shorter than the second edge.

Example 28. The prosthetic valve of any example herein, particularly any one of examples 23-27, wherein each commissure tab extends through a corresponding commissure window of the frame, and wherein the notches space the coaptation edge regions of the leaflets radially inwardly away from an inner surface of the frame.

Example 29. A prosthetic valve, comprising: a radially expandable and compressible frame comprising an inflow end and an outflow end; and a leaflet assembly comprising a plurality of leaflets coupled to the frame, wherein each leaflet comprises an outflow edge, a cusp edge opposite to the outflow edge, and a pair of commissure tabs extending from opposite sides of the leaflet and between the outflow edge and the cusp edge, wherein each commissure tab is paired with an adjacent commissure tab of an adjacent leaflet to form a commissure of the leaflet assembly that is connected to the frame, wherein the outflow edge of each leaflet comprises a coaptation edge region and chamfered or rounded edges connecting the coaptation edge region to the corresponding commissure tabs.

Example 30. The prosthetic valve of any example herein, particularly example 29, wherein each commissure tab extends through a corresponding commissure window of the frame, wherein the chamfered or rounded edges space the coaptation edge regions of the leaflets radially inwardly away from an inner surface of the frame.

Example 31. The prosthetic valve of any example herein, particularly any one of examples 29-30, wherein for each leaflet, the commissure tabs are axially offset from the coaptation edge region toward the inflow end by the chamfered or rounded edges.

Example 32. The prosthetic valve of any example herein, particularly example 31, wherein for each leaflet, the chamfered or rounded edges extend at obtuse angles relative to the coaptation edge region.

Example 33. The prosthetic valve of any example herein, particularly any one of examples 29-32, wherein at each commissure, a primary pivot axis extends through an intersection of a chamfered or rounded edge and an outflow edge of a commissure tab and a secondary pivot axis is radially outward of the primary pivot axis such that at each commissure, the coaptation edge regions of adjacent leaflets pivot at respective primary pivot axes when the leaflets move from a close state to a partially opened state and commissure tabs of adjacent leaflets pivot at respective secondary pivot axes when the leaflets move from the partially opened state to a fully opened state.

Example 34. The prosthetic valve of any example herein, particularly any one of examples 29-33, wherein each commissure tab has exactly one layer of leaflet material.

Example 35. The prosthetic valve of any example herein, particularly any one of examples 29-34, wherein each pair of commissure tabs extends through a commissure window of the frame.

Example 36. A prosthetic valve, comprising: a radially expandable and compressible frame comprising an inflow end and an outflow end; and a leaflet assembly comprising a plurality of leaflets coupled to the frame, wherein each leaflet comprises an outflow edge, a cusp edge opposite to the outflow edge, and a pair of commissure tabs extending from opposite sides of the leaflet and between the outflow edge and the cusp edge, wherein the outflow edge of each leaflet comprises a coaptation edge region and cut-out regions between opposite ends of the coaptation edge region and the commissure tabs, wherein each commissure tab comprises an outflow edge portion which extends closer to the outflow end than the coaptation edge region of the corresponding leaflet.

Example 37. The prosthetic valve of any example herein, particularly example 36, wherein the cut-out regions space the coaptation edge regions of the leaflets radially inwardly away from an inner surface of the frame.

Example 38. The prosthetic valve of any example herein, particularly any one of examples 36-37, wherein each commissure tab comprises an outflow edge configured to be inserted into a corresponding commissure window of the frame.

Example 39. The prosthetic valve of any example herein, particularly example 38, wherein the outflow edge portion of each commissure tab comprises a rounded edge connecting the outflow edge of the commissure tab to the corresponding cut-out region.

Example 40. The prosthetic valve of any example herein, particularly any one of examples 38-39, wherein at least a portion of the outflow edge portion is inserted into the corresponding commissure window of the frame.

Example 41. The prosthetic valve of any example herein, particularly example 38, wherein each commissure tab has a side edge connected to and oriented perpendicular to the outflow edge of the commissure tab.

Example 42. The prosthetic valve of any example herein, particularly example 41, wherein the side edge abuts an inner surface of the corresponding commissure window.

Example 43. The prosthetic valve of any example herein, particularly any one of examples 38-42, wherein when a leaflet is flattened, the outflow edge of each commissure tab of the leaflet is angled relative to the corresponding coaptation edge region.

Example 44. The prosthetic valve of any example herein, particularly any one of examples 38-42, wherein when a leaflet is flattened, the outflow edge of each commissure tab is parallel to the corresponding coaptation edge region.

Example 45. The prosthetic valve of any example herein, particularly any one of examples 36-44, wherein each cut-out region comprises a notch.

Example 46. The prosthetic valve of any example herein, particularly example 45, wherein each notch is continuously curved from the outflow edge portion to the coaptation edge region.

Example 47. The prosthetic valve of any example herein, particularly example 46, wherein each notch has a concave curvature.

Example 48. The prosthetic valve of any example herein, particularly example 45, wherein each notch comprises a first edge extending from the outflow edge portion to a nadir of the notch and a second edge extending from the nadir to the coaptation edge region.

Example 49. The prosthetic valve of any example herein, particularly example 48, wherein the first edge is curved.

Example 50. The prosthetic valve of any example herein, particularly example 49, wherein the first edge has a concave curvature.

Example 51. The prosthetic valve of any example herein, particularly any one of examples 48-50, wherein the second edge is straight.

Example 52. The prosthetic valve of any example herein, particularly example 48, wherein the first and second edges are straight.

Example 53. The prosthetic valve of any example herein, particularly any one of examples 36-52, wherein each leaflet has primary pivot axes extending through the cut-out regions and a secondary pivot axes radially outward of the primary pivot axes such that the coaptation edge regions of adjacent leaflets pivot at respective primary pivot axes when the leaflets move from a close state to a partially opened state and commissure tabs of adjacent leaflets pivot at respective secondary pivot axes when the leaflets move from the partially opened state to a fully opened state.

Example 54. The prosthetic valve of any example herein, particularly example 53, wherein each commissure tab is separated from the cusp edge by a recessed region of the leaflet.

Example 55. The prosthetic valve of any example herein, particularly example 54, wherein each primary pivot axis extends from a nadir of a cut-out region to a nadir of the recessed region.

Example 56. The prosthetic valve of any example herein, particularly any one of examples 36-55, wherein each commissure tab has exactly one layer of leaflet material.

Example 57. A method of assembling a prosthetic valve, the method comprising: receiving a radially expandable and compressible frame comprising an inflow end and an outflow end; receiving a plurality of leaflets, wherein each leaflet comprises an outflow edge, a cusp edge opposite to the outflow edge, and a pair of commissure tabs extending from opposite sides of the leaflet and between the outflow edge and the cusp edge, wherein the outflow edge of each leaflet comprises a coaptation edge region and cut-out regions between opposite ends of the coaptation edge region and the commissure tabs, forming a leaflet assembly by pairing each commissure tab with an adjacent commissure tab of an adjacent leaflet to create a corresponding commissure; and attaching the commissures to the frame, wherein the attaching maintains the cut-out regions within an interior space of the frame so that the coaptation edge regions are spaced radially apart from an inner surface of the frame.

Example 58. The method of any example herein, particularly example 57, wherein the attaching comprises inserting the commissures through corresponding commissure windows of the frame.

Example 59. The method of any example herein, particularly example 58, wherein the attaching comprises suturing a wedge member between the commissure tabs of each commissure at a location outside the frame.

Example 60. The method of any example herein, particularly any one of examples 57-59, further comprising suturing the cusp edge of each leaflet to struts of the frame.

Example 61. The method of any example herein, particularly example 60, further comprising aligning alignment features on the cusp edge of each leaflet with adjacent struts of the frame.

Example 62. The method of any example herein, particularly example 61, wherein the alignment features comprise one or more notches on the cusp edge of each leaflet and/or one or more projections on the cusp edge of each leaflet.

Example 63. A method of assembling a prosthetic valve, the method comprising: receiving a radially expandable and compressible frame comprising an inflow end and an outflow end; receiving a leaflet assembly comprising a plurality of leaflets, each leaflet comprising an outflow edge and a cusp edge; aligning one or more alignment features on the cusp edge of each leaflet with one or more adjacent portions of the frame; and suturing the cusp edge of each leaflet to the frame.

Example 64. The method of any example herein, particularly example 63, wherein the alignment features comprise one or more notches on the cusp edge of each leaflet and/or one or more projections on the cusp edge of each leaflet.

Example 65. The method of any example herein, particularly example 64, wherein the alignment features comprise one or more triangular projections extending from the cusp edge of each leaflet.

Example 66. The method of any example herein, particularly example 65, wherein the one or more triangular projections comprise two triangular projections on opposite sides of each leaflet.

Example 67. The method of any example herein, particularly any one of examples 63-66, wherein the act of aligning alignment features on the cusp edge of each leaflet comprises aligning an alignment feature at an apex region of the cusp edge of each leaflet with an inflow apex of the frame.

Example 68. The method of any example herein, particularly example 67, wherein the alignment feature at the apex region of the cusp edge of each leaflet comprises a notch.

Example 69. The method of any example herein, particularly any one of examples 63-67, wherein the act of aligning alignment features on the cusp edge of each leaflet comprises aligning two alignment features of each leaflet with respective struts of the frame that are spaced equidistant from the inflow end of the frame.

Example 70. The method of any example herein, particularly example 69, wherein the two alignment features of each leaflet aligned with respective struts comprises projections.

Example 71. The method of any example herein, particularly any one of examples 63-70, wherein each leaflet comprises a pair of commissure tabs extending from opposite sides of the leaflet and between the outflow edge and the cusp edge, wherein the outflow edge of each leaflet comprises a coaptation edge region and cut-out regions between opposite ends of the coaptation edge region and the commissure tabs, wherein each commissure tab is paired with a commissure tab of an adjacent leaflet to form a commissure, and wherein the method further comprises: attaching the commissures to the frame such that the cut-out regions are within an interior space of the frame and that the coaptation edge regions are spaced radially apart from an inner surface of the frame.

Example 72. The method of any example herein, particularly example 71, wherein the act of attaching the commissures to the frame comprises inserting the commissures through corresponding commissure windows of the frame.

Example 73. A prosthetic valve, comprising: a radially expandable and compressible frame comprising an inflow end and an outflow end; and a leaflet assembly comprising a plurality of leaflets coupled to the frame, wherein each leaflet comprises an outflow edge, a cusp edge opposite to the outflow edge, and a pair of commissure tabs extending from opposite sides of the leaflet and between the outflow edge and the cusp edge, wherein each commissure tab is paired with an adjacent commissure tab of an adjacent leaflet to form a commissure of the leaflet assembly that is connected to the frame, wherein the outflow edge of each leaflet comprises a coaptation edge region and cut-out regions between opposite ends of the coaptation edge region and the commissure tabs, wherein under a forward flow of blood through the leaflet assembly, the coaptation edge regions pivot relative to the commissure tabs at respective primary pivot axes intersecting the cut-out regions to move the leaflet assembly from a closed state to a partially opened state, after which the commissure tabs pivot relative to each other at respective secondary pivot axes to move the leaflets from the partially opened state to a fully opened state.

Example 74. The prosthetic valve of any example herein, particularly example 73, wherein each commissure tab has exactly one layer of leaflet material.

Example 75. The prosthetic valve of any example herein, particularly any one of examples 73-74, wherein the cut-out regions comprise notches formed in the outflow edge of each leaflet.

Example 76. The prosthetic valve of any example herein, particularly example 75, wherein each primary pivot axis extends through a nadir of a corresponding notch.

Example 77. The prosthetic valve of any example herein, particularly example 76, wherein each notch comprises a first edge extending between the nadir and a corresponding coaptation edge region, wherein the first edge and the corresponding coaptation edge region form an angle of 90 degrees or greater.

Example 78. The prosthetic valve of any example herein, particularly example 77, wherein each notch comprises a second edge extending between the nadir and an outflow edge of a corresponding commissure tab, wherein the second edge and the outflow edge of the corresponding commissure tab form an obtuse angle.

Example 79. The prosthetic valve of any example herein, particularly example 78, wherein when a leaflet is flattened, the outflow edge of each commissure tab of the leaflet is angled relative to the corresponding coaptation edge region.

Example 80. The prosthetic valve of any example herein, particularly example 73, wherein each commissure tab comprises an outflow edge that extends closer to the outflow end of the frame than the coaptation edge region of the corresponding leaflet.

Example 81. The prosthetic valve of any example herein, particularly example 80, wherein the outflow edge of each commissure tab comprises a protruding portion.

Example 82. The prosthetic valve of any example herein, particularly example 81, wherein the protruding portion has a rounded edge connecting an outflow edge of the commissure tab to a corresponding cut-out region.

Example 83. The prosthetic valve of any example herein, particularly any one of examples 81-82, wherein at least a portion of the protruding portion is inserted into a corresponding commissure window of the frame.

Example 84. The prosthetic valve of any example herein, particularly example 73, wherein the cut-out regions comprise chamfered edges formed in the outflow edge of each leaflet.

Example 85. The prosthetic valve of any example herein, particularly example 73, wherein the cut-out regions comprise rounded edges formed in the outflow edge of each leaflet.

Example 86. The prosthetic valve of any example herein, particularly any one of examples 73-85, wherein each commissure tab is connected to the corresponding cusp edge by a recessed region.

Example 87. The prosthetic valve of any example herein, particularly any one of examples 73-86, wherein each leaflet comprises exactly two commissure tabs.

Example 88. A method, comprising: delivering a prosthetic device in a radially compressed state to a target location; and radially expanding the prosthetic device to a radially expanded state, wherein the prosthetic device is a prosthetic valve according to any one of examples 1-56 and 73-87.

Example 89. A method comprising sterilizing the prosthetic valve of any one of examples 1-56 and 73-87.

The features described herein with regard to any example can be combined with other features described in any one or more of the other examples, unless otherwise stated. For example, any one or more of the features of one prosthetic valve can be combined with any one or more features of another prosthetic valve.

In view of the many possible examples to which the principles of the disclosed technology may be applied, it should be recognized that the illustrated examples are only preferred examples of the technology and should not be taken as limiting the scope of the disclosure. Rather, the scope of the claimed subject matter is defined by the following claims and their equivalents.

What is claimed is:

1. A prosthetic valve, comprising:
a radially expandable and compressible frame comprising an inflow end and an outflow end; and
a leaflet assembly comprising a plurality of leaflets coupled to the frame,
wherein the leaflet assembly is movable between an open state which permits blood flow from the inflow end to the outflow end and a closed state which blocks blood fluid flow from the outflow end to the inflow end,
wherein each leaflet comprises an outflow edge, a cusp edge opposite to the outflow edge, and a pair of commissure tabs extending from opposite sides of the leaflet and between the outflow edge and the cusp edge, wherein each commissure tab is paired with an adjacent commissure tab of an adjacent leaflet to form a commissure of the leaflet assembly that is connected to the frame,
wherein the outflow edge of each leaflet comprises a coaptation edge region and cut-out regions between opposite ends of the coaptation edge region and the commissure tabs,
wherein when the leaflet assembly moves between the open and closed states, the coaptation edge regions pivot relative to the commissure tabs at respective pivot axes intersecting the cut-out regions.

2. The prosthetic valve of claim 1, wherein the cut-out regions comprise notches formed in the outflow edge of each leaflet.

3. The prosthetic valve of claim 2, wherein each pivot axis extends through a nadir of a corresponding notch.

4. The prosthetic valve of claim 3, wherein each notch comprises a first edge extending between the nadir and a corresponding coaptation edge region, wherein the first edge and the corresponding coaptation edge region form an angle of 90 degrees or greater.

5. The prosthetic valve of claim 4, wherein each notch comprises a second edge extending between the nadir and an outflow edge of a corresponding commissure tab, wherein the second edge and the outflow edge of the corresponding commissure tab form an obtuse angle.

6. The prosthetic valve of claim 1, wherein each commissure tab comprises a protruding portion which extends closer to the outflow end than the coaptation edge region of the corresponding leaflet.

7. The prosthetic valve of claim 6, wherein the protruding portion has a rounded edge connecting an outflow edge of the commissure tab to a corresponding cut-out region.

8. The prosthetic valve of claim 7, wherein at least a portion of the protruding portion is inserted into a corresponding commissure window of the frame.

9. The prosthetic valve of claim 6, wherein the protruding portion has a side edge connected to and oriented perpendicular to an outflow edge of the commissure tab.

10. The prosthetic valve of claim 9, wherein the outflow edge of the commissure tab extends through a corresponding commissure window of the frame, wherein the side edge abuts an inner surface of the corresponding commissure window.

11. The prosthetic valve of claim 1, wherein the cut-out regions comprise chamfered edges formed in the outflow edge of each leaflet.

12. The prosthetic valve of claim 1, wherein the cut-out regions comprise rounded edges formed in the outflow edge of each leaflet.

13. The prosthetic valve of claim 11, wherein each commissure tab extends through a corresponding commissure window of the frame, and wherein the cut-out regions space the coaptation edge regions of the leaflets radially inwardly away from an inner surface of the frame.

14. The prosthetic valve of claim 1, wherein portions of the commissure tabs at each commissure are configured to pivot relative to each other when the leaflet assembly moves between the open and closed states.

15. The prosthetic valve of claim 14, wherein when the leaflet assembly moves from the closed state to the open state, the coaptation edge regions move apart from each other, after which the commissure tabs at each commissure move apart from each other.

16. A prosthetic valve, comprising:
a radially expandable and compressible frame comprising an inflow end and an outflow end; and
a leaflet assembly comprising a plurality of leaflets coupled to the frame,
wherein each leaflet comprises an outflow edge, a cusp edge opposite to the outflow edge, and a pair of commissure tabs extending from opposite sides of the leaflet and between the outflow edge and the cusp edge,
wherein the outflow edge of each leaflet is formed with a notch adjacent each commissure tab.

17. The prosthetic valve of claim 16, wherein the outflow edge of each leaflet defines a coaptation edge region extending between the notches.

18. The prosthetic valve of claim 17, wherein each commissure tab extends through a corresponding commissure window of the frame, and wherein the notches space the coaptation edge regions of the leaflets radially inwardly away from an inner surface of the frame.

19. A prosthetic valve, comprising:
a radially expandable and compressible frame comprising an inflow end and an outflow end; and
a leaflet assembly comprising a plurality of leaflets coupled to the frame,
wherein each leaflet comprises an outflow edge, a cusp edge opposite to the outflow edge, and a pair of commissure tabs extending from opposite sides of the leaflet and between the outflow edge and the cusp edge, wherein each commissure tab is paired with an adjacent commissure tab of an adjacent leaflet to form a commissure of the leaflet assembly that is connected to the frame,
wherein the outflow edge of each leaflet comprises a coaptation edge region and cut-out regions between opposite ends of the coaptation edge region and the commissure tabs,
wherein under a forward flow of blood through the leaflet assembly, the coaptation edge regions pivot relative to the commissure tabs at respective primary pivot axes intersecting the cut-out regions to move the leaflet assembly from a closed state to a partially opened state, after which the commissure tabs pivot relative to each other at respective secondary pivot axes to move the leaflets from the partially opened state to a fully opened state.

20. The prosthetic valve of claim 19, wherein each commissure tab has exactly one layer of leaflet material.
